(19) United States
(12) Patent Application Publication
(10) Pub. No.: US 2026/0275390 A1
(43) Pub. Date: Sep. 17, 2026

(54) PRODUCTION AND PURIFICATION OF NATURAL VANILLIN USING NEW STRAINS OF AMYCOLATOPSIS

(71) Applicant: SPERO RENEWABLES, LLC, Goleta, CA (US)

(72) Inventors: Ian M. KLEIN, Goleta, CA (US); Shou ZHAO, Goleta, CA (US); Baoyuan LIU, Goleta, CA (US); Sean PRAGER, Goleta, CA (US)

(21) Appl. No.: 19/167,287

(22) PCT Filed: Mar. 21, 2024
(86) PCT No.: PCT/US2024/020995

§ 371 Date: Sep. 19, 2025

Related U.S. Application Data
(60) Provisional application No. 63/491,486, filed on Mar. 21, 2023.

(51) Int. Cl. C12P 7/24; C12N 1/20

(52) U.S. Cl. CPC C12P 7/24; C12N 1/20; C12N 2501/999

(57) ABSTRACT

A composition includes a biologically pure culture of Amycolatopsis tucumanensis LGM 24814, a mutant thereof, or an enzyme extracted thereof, ferulic acid, and a carbon source. Also disclosed is a process for preparing vanillin that includes subjecting ferulic acid to Amycolatopsis tucumanensis LGM 24814, a mutant thereof, or an isolated enzyme thereof for a period of time sufficient to convert the ferulic acid to vanillin, converting at least a portion of the ferulic acid to vanillin, and recovering the vanillin.

### CROSS REFERENCE TO RELATED APPLICATIONS

This application claims priority to U.S. Provisional Patent Application No. 63/491,486 filed on Mar. 21, 2023, and entitled “PRODUCTION AND PURIFICATION OF NATURAL VANILLIN USING NEW STRAINS OF AMYCOLATOPSIS,” which is incorporated herein in its entirety by reference.

### BACKGROUND

Vanillin, of the chemical name 4-hydroxy-3-methoxybenzaldehyde, is one of the most important aromatic flavor compounds used in foods, beverages, fragrances, pharmaceuticals and polymers. Consumer demands for natural products and ingredients have driven the flavor industry to seek new sources of natural vanillin, while vanillin derived from synthetic, non-natural sources such as petrochemicals and eugenol have seen a decrease in demand. Vanilla extracts obtained from the vanilla bean cannot keep pace with rising demand and human population; it is limited to certain geographic locations and results in harmful deforestation. As a result, additional sources of vanillin would be useful.

### FIELD OF THE DISCLOSURE

The present disclosure generally relates to non-genetically modified microorganisms suitable for the production of natural vanillin using renewable feedstock of varying purities and purification of the resulting vanillin product. More specifically, the present processes disclose that a specific strain of Amycolatopsis, Amycolatopsis tucumanensis (LGM 24814; DSM 45259; JCM 17017; Trujillo ABO), which has not previously been reported in vanillin production can be used to convert ferulic acid to vanillin in high yields.

### SUMMARY

In some embodiments, a composition comprises a biologically pure culture of Amycolatopsis tucumanensis LGM 24814, a mutant thereof, or an enzyme extracted thereof; ferulic acid; and a carbon source.

In some embodiments, a process for preparing vanillin comprises: subjecting ferulic acid to Amycolatopsis tucumanensis LGM 24814, a mutant thereof, or an isolated enzyme thereof for a period of time sufficient to convert the ferulic acid to vanillin; converting at least a portion of the ferulic acid to vanillin; and recovering the vanillin.

In some embodiments, a process for preparing vanillin comprising subjecting ferulic acid to Amycolatopsis tucumanensis LGM 24814, a mutant thereof, or an isolated enzyme thereof for a period of time sufficient to convert the ferulic acid to vanillin, converting ferulic acid to vanillin, and recovering the vanillin. The Amycolatopsis tucumanensis LGM 24814 or mutant thereof is contained in a medium which comprises a carbon source.

In some embodiments, a process for preparing vanillin comprises subjecting ferulic acid to Amycolatopsis tucumanensis LGM 24814, a mutant thereof, or an isolated enzyme thereof which converts ferulic acid to vanillin, for a period of time sufficient to convert the ferulic acid to vanillin, and recovering the vanillin. The Amycolatopsis tucumanensis LGM 24814 or mutant thereof is contained in a medium which comprises a nitrogen source.

In some embodiments, a process for preparing vanillin comprises subjecting ferulic acid to Amycolatopsis tucumanensis LGM 24814, a mutant thereof, or an isolated enzyme thereof which converts ferulic acid to vanillin, for a period of time sufficient to convert the ferulic acid to vanillin, and recovering the vanillin, wherein the Amycolatopsis tucumanensis LGM 24814 or mutant thereof is contained in a medium which comprises antifoam agent (deformer).

In some embodiments, a process for preparing vanillin comprises subjecting ferulic acid to Amycolatopsis tucumanensis LGM 24814, a mutant thereof, or an isolated enzyme thereof which converts ferulic acid to vanillin, for a period of time sufficient to convert the ferulic acid to vanillin, and recovering the vanillin thus formed, wherein the Amycolatopsis tucumanensis LGM 24814 or mutant thereof is contained in a medium which comprises dissolved oxygen content.

In some embodiments, a process for preparing vanillin comprises subjecting ferulic acid to the microorganism Amycolatopsis tucumanensis LGM 24814, a mutant thereof, or an isolated enzyme thereof which converts ferulic acid to vanillin, converting the ferulic acid to the vanillin in a concentration of the vanillin of at least 1 g/l within a period of time from 4 to 120 hours, and recovering the vanillin thus formed.

In some embodiments, a process comprises obtaining a fermentation broth comprising vanillin in water, and impurities, and performing the following steps: a) a step of centrifugation of fermentation broth to remove cellular biomass/debris and particulate matter, b) a step of liquid/liquid extraction by placing the aqueous solution obtained after step a) in contact with a solvent E7, to obtain an organic phase containing vanillin and impurities, and an aqueous phase containing water-soluble impurities, salts, and residual solvent, c) a step concentrating the vanillin rich organic phase to yield a concentrated crude vanillin product, d) a step of distillation of the crude concentrated vanillin obtained from the evaporation of the organic phase in step c), and e) a step of multi-step recrystallization of the vanillin to yield a high purity vanillin.

In some embodiments, a process for purifying vanillin and derivatives thereof, starting with an initial solution of vanillin or of a vanillin derivative in a solvent S1 containing impurities, comprising the following steps: evaporating the solvent S1 in the presence of water from the initial solution of the vanillin or of the vanillin derivative containing the solvent S1 to obtain an aqueous solution of the vanillin or of the vanillin derivative, using liquid/liquid extraction by placing the aqueous solution obtained after step a) in contact with a solvent S2, at a pH greater than 8 and less than 10, to obtain an organic phase comprising the solvent S2 and an aqueous phase containing the vanillin or the vanillin derivative and residual solvent S2, precipitating, at a pH of between 4 and 7.5, the Vanillin or the derivative contained in the aqueous phase obtained after the liquid-liquid extraction, and isolating the vanillin or the derivative thereof.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 illustrates an embodiment of a purification process using a stripping approach.

FIG. 2 Illustrates an embodiment of a vanillin purification method.

### DETAILED DESCRIPTION

The most viable solution to obtaining natural vanillin is through fermentation of natural ferulic acid which can be sourced from a suitable natural/biomass feedstock. Carbon 13 NMR of vanillin (the active molecule in vanilla flavoring) can be used to determine the source of vanillin by the ratio of ¹³C and ¹²C at the eight carbons of the vanillin molecule, with the carbons of the aldehyde and the methoxy group determined to be the most important. Synthetic sources such as guaiacol can be discriminated from natural vanillin sourced from vanilla beans and ferulic acid, with thresholds dependent on the experimental setup.

A variety of biochemical processes for the production of vanillin have been developed based on the conversion of the following substrates: lignin, phenolic stilbenes, isoeugenol, eugenol, ferulic acid, sugars, vanillic acid, waste residues and aromatic amino acids under the catalysis of specific microorganisms. A review from Kaur and Chakrabborty (Kaur B, Chakraborty D. “Biotechnological and molecular approaches for vanillin production: a review.” Appl. Biochem Biotechnol. 2013 February; 169(4): 1353-72) lists several biosynthetic pathways and known microorganisms for the conversion of ferulic acid to vanillin.

Processes for the conversion of ferulic acid to vanillin using two novel species of the genus Amycolatopsis which were deposited at the Deutsche Sammlung fur 7 Mikroorganismen und Zellkulturen GmbH in Brunswick under the numbers DSM 991 and DSM 9992 were reported by Rabenhorst et al. [US006133003A]. The actinomycete Amycolatopsis sp. Strain ATCC 39116 has been used for the conversion of ferulic acid to vanillin as referenced in (Kaur B, Chakraborty D. “Biotechnological and molecular approaches for vanillin production: a review.” Appl. Biochem Biotechnol. 2013 February; 169(4): 1353-72). Further reference to ferulic acid conversion to vanillin using Amycolatopsis sp. Strain ATCC 39116 is found in [WO2022/133254A1] and [U.S. Pat. No. 6,235,507B1]. The complete genetic sequence of Amycolatopsis sp. Strain ATCC39116 has been published and the information is accessible in NCBI under accession number J11414689.1, and in Genbank under accession number AFWY00000000 (Davis J. R., Goodwin L. A., Woyke T., Teshima H., Bruce D., Defter C., Tapia R., Han S., Pitluck S., Nolan M., Mikhailova N., Land M. L. and Sello J. K., Genome sequence of Amycolatopsis sp. strain ATCC 39116, a plant biomass-degrading actinomycete J. Bacteriology, 2012, vol. 194, 2396)

A review from Veena Paul et al (Paul, V., et al. “A comprehensive review on vanillin: its microbial synthesis, isolation and recovery”. Food Biotechnology 2021. 35:1, 22-49) summarizes work in which Amycolatopsis strain ATCC 39116 has been genetically modified to improve the yield of vanillin produced from ferulic acid by minimizing or eliminating metabolic routes for the further conversion of vanillin to vanillic acid and/or vanillyl alcohol. However, many consumers have shown a preference for vanillin which has been produced by wild-type non-genetically modified organisms.

Amycolatopsis tucumanensis (LGM 24814) is an actinobacterial strain isolated from copper containing groundwater sediments in Tucuman Argentina (Albarracin, 2005). Amycolatopsis tucumanensis was deposited in the Belgian Coordinated Collections of Microorganisms (BCCM) with a species name of: Amycolatopsis tucumanensis Albarracin, Alsono-Vega, Trujillo, Amoroso and Abate 2010 VP and strain number LMG 24814. The Amycolatopsis tucumanensis strain is also referred to by the collection numbers of LGM 24814, DSM 452259, JCM 17017, and Trujillo ABO. Further genotypic and phenotypic data of the Amycolatopsis tucumanensis discovered by Albarracin et al., confirmed that Amycolatopsis tucumanensis (DSM 45259, LGM 24814) represents a novel species in the genus Amycolatopsis (Albarracin et al 2010).

Complete genome sequencing and genomic analysis of Amycolatopsis tucumanensis (LGM 24814, DSM 45259) was completed in 2022 (Guerrero et al 2022). The Whole Genome Shotgun project of Amycolatopsis tucumanensis has been deposited at DDBJ/ENA/GenBank under the accession JAKGSD000000000 (Guerrero et al 2022). To date, there have been no reports on the conversion of ferulic acid to vanillin using Amycolatopsis tucumanensis (LGM 24814, DSM 45259).

The present disclosure relates to a fermentative process for the preparation of vanillin from ferulic acid and to microorganisms suitable for this process. The process and systems relate to a novel species of the genus Amycolatopsis, Amycolatopsis tucumanensis (LGM 24814, DSM 45259) which can be used to convert ferulic acid to vanillin. This disclosure further relates to a process for the preparation of vanillin from ferulic acid in the presence of Amycolatopsis tucumanensis or their enzymes or of microorganisms having genetic material from Amycolatopsis tucumanensis, which genetic material codes for the structural and regulatory genes for the enzymes which are active in this reaction. This disclosure further relates to the purification of the resulting vanillin product and its use in any application where vanillin is used.

The organism can be cultured in a conventional culture medium in a conventional manner for the culturing of microorganisms. The substrate can be added at the beginning of the incubation, during or after completion of growth, all at once or distributed over a relatively long period. The amount of ferulic acid is advantageously of a magnitude such that the concentration of the compound in the culture broth does not exceed about 80 g/L. The course of the reaction can be followed by determining the starting material and the product in the culture broth by high performance liquid chromatography (HPLC). After the desired amount of vanillin has formed, this is isolated from the culture broth by known physical methods such as extraction, distillation or chromatography, which are described in more detail herein. The crude product thus obtained can be purified by further steps.

The microorganism of the disclosure can be cultured in synthetic, semisynthetic, or complex culture media. These culture media contain carbon sources, nitrogen sources, inorganic salts and, if appropriate, trace elements and vitamins. Carbon sources which can be used can include, but are not limited to, sugars such as glucose, sugar alcohols such as glycerol or mannitol, organic acids such as citric acid, and/or complex mixtures such as malt extract, yeast extract, casein or casein hydrolysate.

Examples of suitable nitrogen sources include but are not limited to inorganic nitrogen sources such as nitrates and ammonium salts and organic nitrogen sources such as yeast extract, soybean meal, cottonseed meal, casein, casein hydrolysate, wheat gluten and corn Steep liquor. Inorganic salts which can be used include but are not limited to, for example, sulfates, nitrates, chlorides, carbonates and phosphates of sodium, potassium, magnesium, calcium, zinc, and/or iron.

The culture temperature is preferably in the range from about 10 to about 75° C., particularly in the range from about 25 to about 50° C. The pH of the medium can be in a range of 3 to 11, in particular 4 to 9. The microorganisms can be cultured either in suitable shaking apparatuses or in fermenters equipped with a stirrer device. Care must be taken to ensure adequate aeration in culturing. In some aspects, the dissolved oxygen content in the medium can be maintained between 0-25 mg oxygen per liter of medium. The dissolved oxygen content in medium can be maintained by 0-20 Hz (0-1200 rpm) agitation.

In some aspects, the fermentation with the ferulic acid can be carried out with the medium comprising an antifoaming agent (e.g., a defoamer). Any suitable antifoaming agent can be used. In some aspects, the antifoam agent can be an organic non-silicon-based polymer, liquid silicon-based polymer, solid non-ionic polymer, fatty acids and neutral lipids natural oil, mixture of natural fatty acids and triacylglycerols, or any combination thereof. The antifoam agent can be present in the medium with Amycolatopisis tucumanensis LGM 24814, or mutant thereof, or isolated enzyme thereof at concentration between 0.1 ppm to 300 ppm.

The microorganisms can be cultured batchwise, semi continuously or continuously. The culture time until a desired amount of product has been achieved is between 4 and 120 hours after inoculation of the culture. To protect the microorganisms from the toxic activity of the substances used or formed, it can be advantageous to add adsorbents to the culture media, e.g. activated carbon or adsorber resins such as Amberlite XAD-2, Amberlite XAD-7, Amberlite XAD-16, Amberlite XAD-16N, Lewatit OC 1062, Lewatit OC 1064, polystyrene divinylbenzene resins, cation exchange resins, or anion exchange resins. To protect the microorganisms from the toxic activity of the substances used or formed, it can be advantageous to perform continuous or semi-continuous extraction of substances used or formed to remove them from the culture/fermentation media.

In some embodiments Amycolatopsis tucumanensis LGM 24814 is cultured in a batch, semi continuous or continuous culture system. A substrate of ferulic acid is added to the culture system for conversion to the desired vanillin product. Adsorbents such as activated carbon or adsorber resins such as Amberlite XAD-2, Amberlite XAD-7, Amberlite XAD-16, Amberlite XAD-16N, Lewatit OC 1062, Lewatit OC 1064, polystyrene divinylbenzene resins, cation exchange resins, or anion exchange resins or similar resins can optionally be added to the culture system to adsorb and remove products or reagents from the solution. In some embodiments the added activated carbon or adsorber resins exhibit the ability to preferentially bind vanillin compared to ferulic acid. The activated carbon or adsorber resins when added to the culture system can selectively bind the vanillin product in culture media, effectively removing the vanillin product from the culture media by binding it to the solid resin. Vanillin, ferulic acid, or other products from the culture media which are bound or adsorbed by the resin can be desorbed or released from the resin by washing the resin with a desorbing solvent including but not limited to aliphatic alcohols, water, mixtures of water and a dissolved base, or combinations thereof. Advantageously, selective removal of vanillin product from the culture media can prevent further conversion of vanillin to undesired products including but not limited to vanillyl alcohol, vanillic acid, and guaiacol. Furthermore, as certain concentrations of vanillin and other compounds are known to be toxic to microorganisms such as Amycolatopsis, selective removal of vanillin and other compounds from the culture media can protect the microorganism from the toxic activity of the substances used or formed.

In some embodiments a 2-phase partitioning bio-reactor system can be used to selectively remove vanillin products from the culture media. In some embodiments a 2-phase reactor uses a liquid-liquid separation to remove vanillin products from the culture media.

In some embodiments, the ferulic acid feed which is converted to vanillin using Amycolatopsis tucumanensis LGM 24814, is produced from an extraction of biomass. Biomass extracts containing ferulic acid can optionally undergo a range of purification steps to produce a ferulic acid product where the purity of the ferulic acid product can range from 1 wt. % pure ferulic acid to 100 wt. % pure ferulic acid. When the ferulic acid product has a purity of less than 100 wt. % purity, the contaminants can be grouped into four categories of Group 1, Group 2, Group 3, and Group 4 contaminants as follows. Group 1 contaminants can comprise lignin or lignin derived materials and can include any combination of, some of, or one of the following: lignin, polyphenols, coumaric acid, substituted phenols, or lignin derived materials. While not wishing to be limited by theory, it is noted that Group 1 contaminants can often be characterized by UV-Visible Spectroscopy and often have a Lamda max of about 200 nanometers (nm) and a local maximum absorbance at about 240 nm or about 320 nm, or Group 1 contaminants can be assayed as Total Phenolic Content using several methods. Group 2 contaminants can comprise carbohydrates or carbohydrate derived materials and can include any combination of, some of, or one of the following: carbohydrates, hemicellulose, cellulose, arabinoxylans, heteroxylans, xylans, glucans, sugars, and carbohydrate derived materials. Group 3 contaminants can be water soluble and can include any combination, some of, or one of the following: proteins, amino acids, nitrogen containing compounds, and salts. Group 4 contaminants are often characterized as oils and can include any combination of, some of, or one of the following: fatty acids, oleic acid, linoleic acid, fatty acid esters, sterols, and phytosterols. In some embodiments Amycolatopsis tucumanensis LGM 24814 can be used to convert ferulic acid to vanillin in the presence of Group 1-4 contaminants where the purity of the ferulic acid feed is less than 20%, or less than 30%, or less than 50%, or less than 90%, each by weight.

While not wishing to be limited by theory, it is noted that the ≤20% pure ferulic acid described herein can contain 0-70% lignin or lignin derived compounds, 0-60% carbohydrates or carbohydrate derived materials, and 0-30% protein, amino acids, or derivatives of protein as contaminants. When fermented to make vanillin, the resulting product mixture can contain 5-20% vanillin; 0-15% other metabolites including but not limited to guaiacol, vanillic acid, vanillyl alcohol, 4-vinylguaiacol, protocatechuic acid, and catechol; and 0-70% lignin or lignin derived compounds; and 0-60% carbohydrates or carbohydrate derived materials; and 0-30% protein, amino acids, or derivatives of protein.

While not wishing to be limited by theory, it is noted that the ≤30% pure ferulic acid described herein can contain 0-70% lignin or lignin derived compounds and 0-50% carbohydrates, hemicellulose or carbohydrate derived materials as contaminants. When fermented to make vanillin, the resulting product mixture can contain 7.5-30% vanillin; 0-22.5% other bioproducts including but not limited to guaiacol, vanillic acid, vanillyl alcohol, 4-vinylguaiacol, protocatechuic acid, catechol, and ferulic acid; and 0-70% lignin or lignin derived compounds; and 0-50% carbohydrates, hemicellulose or carbohydrate derived materials.

While not wishing to be limited by theory, it is noted that the ≤60% pure ferulic acid described herein can contain 0-40% lignin or lignin derived compounds and 0-40% carbohydrates, hemicellulose or carbohydrate derived materials as contaminants. When fermented to make vanillin, the resulting product mixture can contain 15-60% vanillin; 0-45% other bioproducts including but not limited to guaiacol, vanillic acid, vanillyl alcohol, 4-vinylguaiacol, protocatechuic acid, catechol, ferulic acid; and 0-40% lignin or lignin derived compounds; and 0-40% carbohydrates, hemicellulose or carbohydrate derived materials.

While not wishing to be limited by theory, it is noted that the ≤80% pure ferulic acid described herein can contain ≥20% lignin or lignin derived compounds as contaminants. When fermented to make vanillin, the resulting product mixture can contain 20-80% vanillin; 0-60% other bioproducts including but not limited to guaiacol, vanillic acid, vanillyl alcohol, 4-vinylguaiacol, protocatechuic acid, catechol, and ferulic acid; and 0-20% lignin or lignin derived compounds.

While not wishing to be limited by theory, it is noted that the ≤90% pure ferulic acid described herein can contain ≥10% lignin or lignin derived compounds as contaminants. When fermented to make vanillin, the resulting product mixture can contain 22.5-90% vanillin; 0-67.5% other bioproducts including but not limited to guaiacol, vanillic acid, vanillyl alcohol, 4-vinylguaiacol, protocatechuic acid, catechol, and ferulic acid; and 0-10% lignin or lignin derived compounds.

When ferulic acid of ≤20% purity or up to 60% purity is fermented to make vanillin, it can result in a product mixture product containing 5-60% vanillin; 0-45% bioproducts comprising at least one of guaiacol, vanillic acid, vanillyl alcohol, 4-vinylguaiacol, protocatechuic acid, catechol, and ferulic acid; 0-70% lignin or lignin derived compounds; 0-60% carbohydrates, hemicellulose or carbohydrate derived materials; 0-30% protein, amino acids or protein derivatives; and 0-30% oils, fatty acids, fatty acid esters, sterols or phytosterols.

The present processes and systems thus relate to a process for purifying vanillin and derivatives thereof. Various processes can be used to purify the vanillin. While various processes are described herein, the individual process steps in the processes can be used alone or in various combinations between the processes to produce a purified vanillin and/or a purified starting material for the conversion process of the ferulic acid to vanillin.

In some embodiments, the vanillin can be purified using a process comprising liquid-liquid extraction. In this process and systems, purifying vanillin and derivatives thereof can start with a solution of vanillin or of a vanillin derivative in a solvent S1 containing impurities, comprising the following steps: a) evaporating the solvent S1 in the presence of water to obtain an aqueous solution of vanillin or of a vanillin derivative; b) using liquid/liquid extraction by placing the aqueous solution obtained after step a) in contact with a solvent S2, at a pH between about 8 and about 10, to obtain an organic phase and an aqueous phase containing vanillin or a vanillin derivative and residual solvent S2; c) precipitating, at a pH of between about 4 and about 7.5, the vanillin or a derivative thereof contained in the aqueous phase obtained after step b), and d) isolating the vanillin or a derivative thereof. Further details on each step and options within the steps are provided herein. In some aspects, one or more of steps a)-d) can be omitted to still produce a purified vanillin.

In some embodiments, the process of the present disclosure comprises a step a) that comprises removing (e.g., by evaporation) solvent S1 present in the initial vanillin solution or the solution of the vanillin derivative. In accordance with the process, the evaporation step is performed in the presence of an aqueous solution such as water. Preferably, solvent S1 has a boiling point of less than 100° C., or forms an azeotrope with water having a boiling point of less than 100° C.

Among the solvents S1, examples that may be mentioned include the organic solvents permitted by the regulations, such as alkyl acetates (ethyl acetate, propyl acetate, isopropyl acetate), MEK (methyl ethyl ketone), cyclohexane, dichloromethane, or any combination thereof. Solvent S1 may also be water or comprise some amount of water. Solvent S1 may also be a mixture of organic solvents, especially a mixture of organic solvents mentioned above or a mixture of water and of an organic solvent. According to some embodiments, solvent S1 is ethyl acetate.

In the initial vanillin solution, the weight content of vanillin is between 10% and 60%, from 10% to 40%, or from 10% to 35%, relative to the total weight of the solution. Step a) can comprise removing solvent S1 to obtain an aqueous solution of vanillin in which the weight content of vanillin can be between about 5% and about 40%, or between 5% and 35%, or between 5% and 25% relative to the total weight of the solution.

In accordance with the process, the evaporation Step a) can be performed in the presence of water which is added to the initial vanillin solution before and/or during the implementation of the evaporation step.

According to some embodiments, in the context of step a), solvent S1 of the crude vanillin can be removed by evaporation, for example by distillation or by using an evaporator, in the presence of water, such that the vanillin and the impurities end up in the aqueous phase in soluble or insoluble form. In the case of an evaporation by distillation, solvent S1 may be distilled off at atmospheric pressure or under vacuum or alternatively at atmospheric pressure and then under vacuum.

The water may be added in one or more portions to the initial vanillin solution. It is preferable to use water fit for consumption (for example mains water, reverse osmosis water, distilled water, etc.). It is also possible to use recycled water fit for consumption, originating from the process (for example the washing waters, or the crystallization or precipitation mother liquors), as described herein below.

The amount of water advantageously added to the initial vanillin solution before and/or during the implementation of the evaporation step a) is such that the weight content of vanillin in the aqueous Solution obtained after step a) is advantageously between 5% and 40% by weight, between 5% and 35% by weight, or between 5% and 25% by weight relative to the total weight of the solution. For example, this weight content of the vanillin can be from 10% to 15% by weight of the solution. The aqueous solution of vanillin obtained after the step a) contains the impurities formed during the fermentation and more generally those present in the vanillin solution subjected to the evaporation step. Preferably, the evaporation step a) can be performed at a temperature between 60° C. and 120° C. and more preferably between 80° C. and 120° C.

After step a), the aqueous solution of vanillin or of a vanillin derivative can be subjected to an extraction step, which in some aspect can include a liquid/liquid extraction step, under particular pH conditions. Any other suitable extraction step can also be used. When a liquid-liquid extraction is used, the extraction can be carried out in the presence of a second solvent. The solvent used for the extraction step can be referred to herein below as solvent S2.

This step can be performed at a controlled pH so as to separate certain impurities from vanillin by pKa difference. In particular, the step b) can be performed so as to extract the impurities formed by species with a higher pKa than that of Vanillin. These species can include, for example, vanillyl alcohol, guaiacol and certain dimers, or heavy compounds. The extraction according to step b) may be total or partial. In accordance with step b) of the process, the pH is chosen so as to obtain a high yield of vanillin. For example, the pH can be greater than or including 8 and less than or including 10.

During this step of extraction at controlled pH, the protonated species can be extracted by Solvent S2 and the organic layer predominantly comprises solvent S2. The vanillin then remains in the aqueous phase in the form of vanillate. For the rest of the process, the aqueous phase obtained can be used, e.g., the steps subsequent to the liquid-liquid extraction step can be performed using the aqueous phase containing the vanillate.

According to some embodiments, the extraction solvent S2 can be different from solvent S1 present in the initial Vanillin Solution. According to other embodiments, solvents S1 and S2 can be the similar (e.g., having the same component(s)) or identical. This embodiment is advantageous since it makes it possible in particular to perform only partial evaporation of the solvent according to the step a) of the process and to use the non-evaporated part for performing the extraction (e.g., liquid-liquid extraction) step.

In accordance with the process, solvent S1 and solvent S2 can preferentially be chosen from the solvents permitted by the regulations in force according to directive 2009/32/EC and directive 2010/59/UE concerning extraction solvents used in the manufacture of foodstuffs and ingredients thereof.

In accordance with step b) of the process, the extraction solvent S2 has no or very low to moderate solubility in water. More precisely, the maximum weight content of the solvent S2 in water can be equal to 70 g/L. The solvent S2 can be an organic solvent.

Preferably, solvent S2 is a solvent with low or very low solubility in water, e.g., its maximum weight content in water can be equal to 50 g/L, and preferentially its maximum weight content in water can be equal to 20 g/L. Solvent S2 may also be a water-insoluble solvent. The extraction solvent S2 advantageously has a boiling point less than 200° C., or less than about 150° C. Among the solvents S2 employed for the implementation of step b) of the process, suitable solvents can include, but are not limited so, dichloromethane, cyclohexane, ethyl acetate, propyl acetate, isopropyl acetate, n-butyl acetate, isoamyl acetate, methyl isobutyl ketone (MIBK), butanol, Rhodiasolv RPDE (mixture of dimethyl adipate, dimethyl Succinate and dimethyl glutarate), or any mixture of these solvents.

According to an advantageous embodiment, solvent S2 is chosen from food grade solvents. For example, solvent S2 can be chosen from the group consisting of ethyl acetate, propyl acetate, isopropyl acetate, n-butyl acetate, isoamyl acetate and methyl isobutyl ketone (MIBK), and mixtures thereof. Even more preferably, solvent S2 can be isopropyl acetate.

In order to obtain the particular pH conditions described above, according to one embodiment, step b) of the process comprises the addition of a base, the base possibly being either a weak base or a strong base. Advantageously, the base can be chosen from mineral bases, and more particularly water-soluble mineral bases. In particular, the base can be chosen from the group consisting of alkali metal hydroxides, alkaline-earth metal hydroxides, alkali metal bicarbonates, alkaline-earth metal bicarbonates, alkali metal hydrogen carbonates, alkaline-earth metal hydrogen carbonates, alkali metal phosphates, alkaline-earth metal phosphates, alkali metal hydrogen phosphates and alkaline-earth metal hydrogen phosphates, and mixtures thereof. In some embodiments, the base can be chosen from the following mineral bases: NaOH, KOH and Na₂CO₃. Preferentially, NaOH or KOH is used as base, and more particularly NaOH.

This base addition step makes it possible to adjust the pH for the extraction step to a pH value greater than or including 8 and less than or including 10. Preferably, the extraction step according to step b) of the process is performed at a pH of between 8.1 and 9.5, between 8.3 and 9.5, or between 8.5 and 9. Controlling the pH for the implementation of step b) leads, after the implementation of the process, to the production of a high-purity vanillin with an improved yield.

In accordance with step b) of the process, the addition of the base to the vanillin solution is performed before the addition of solvent S2 or after the addition of solvent S2. According to some embodiments, the base is added rapidly to the vanillin solution, for example in a single portion. According to some embodiments of the process, the base can be added to the vanillin solution, optionally containing solvent S2, at a temperature from 15° C. to 60° C. or preferably between 30° C. and 50° C. According to some embodiments of the process, the base used for performing the step b) can be diluted in water to a concentration from 5% to 30% by weight relative to the weight of the aqueous solution in which the base is diluted.

In accordance with the process, step b) can be performed in a mass ratio between the mass of solvent S2 and the mass of vanillin from about 0.2 to 3, from about 0.5 to 3, or from 0.6 to 1.2.

The extraction step can be performed at atmospheric pressure and at a temperature between 15° C. and 40° C., or between 20° C. and 30° C. In some aspects, the extraction step can be preceded by a cooling step to adjust the temperature of the solution. According to some embodiments, the base can be added to the vanillin solution obtained after step a) at a temperature from 15° C. to 60° C., or from 30° C. to 50° C., and the temperature can then be lowered to a temperature between 15° C. and 40° C., or between 20° C. and 30° C. before adding solvent S2 for the extraction.

After step b), an organic phase and an aqueous phase are obtained. The organic phase can contain some of the impurities, especially the guaiacol, vanillyl alcohol, and dimers, and solvent S2, whereas the aqueous phase contains the vanillin in water, the rest of the impurities, for example ferulic acid, vanillic acid, benzoic acid and/or the particular dimers comprising a ferulic unit, and also residual solvent S2. The solvent S2 present in the organic phase, preferentially freed of impurities, is advantageously recycled upstream of step b).

When the process is conducted batchwise, the organic phase can be allowed to separate out by settling to recover the aqueous phase. When the process is conducted in as a continuous process, the extraction step can be performed by using a series of decanting mixers or at least one stirred, pulsed or packed liquid/liquid extraction column. It may also be performed continuously by using a static mixer and then a centrifugal separator for continuously separating the organic and aqueous phases.

According to some embodiments of the process, the aqueous phase obtained after the extraction step can be subjected to a step for removing residual solvent S2 (“stripping step”) so as to improve the quality of the vanillin obtained after the process. Such a stripping step can be performed under mild conditions, especially by injecting a gaseous fluid (for example water vapor or nitrogen, preferably nitrogen), and/or placing the solution under vacuum in the chamber in which the process is performed. In some aspects, the stripping step can be performed under vacuum. It can be performed at a temperature between 20° C. and 50° C. The duration of this step is, for example, from 40 to 120 minutes.

After step b), the vanillin or the vanillin derivative can be present in aqueous solution in the form of vanillate. The solution can contain a large proportion of the impurities present in the crude vanillin, in particular the species whose pKa is less than that of vanillin. These species are, in particular, ferulic acid, vanillic acid, benzoic acid, and/or particular dimers comprising a ferulic unit. Step c) if the process can be performed at a controlled pH to lower the pH of the solution containing the vanillin in the form of vanillate. By lowering the pH, the Vanillin precipitates out with the impurities remaining in the solution, referred to as the mother liquor. Step c) of the process can be performed at a pH of between 4 and 7.5. These vanillin precipitation conditions make it possible to obtain a suitable yield of vanillin and also a minimum vanillin titer of 97% in the precipitate obtained after the precipitation step. The pH during the precipitation step can be between 5 and 7, between 5.7 and 6.5, or between 5.8 and 6.3.

The precipitation step according to the process can be performed using a weak or strong aqueous acid, which can be introduced into the aqueous phase obtained after step b) of the process. An acid that does not react with vanillin can be used. Among the acids, mention may be made especially of acids whose formed salts are water-soluble. According to some embodiments, the abovementioned precipitation step can be performed in the presence of sulfuric acid.

According to some embodiments of the process, step c) can be performed at a temperature from 15° C. to 40° C., or from 25° C. to 40° C. Advantageously, the precipitation step can be performed at atmospheric pressure. In some embodiments, step c) comprises the addition of an acid, present in aqueous solution, to the aqueous phase obtained by decantation after step b), which can be followed by cooling of the aqueous medium from which the vanillin precipitates. The cooling can be performed to obtain a temperature less than or equal to 20° C., less than or equal to 15° C., or a temperature between 5 and 15° C.

According to some embodiments, the addition of acid to the vanillin aqueous phase obtained from step b) can be performed at a temperature between 50° C. and 95° C. or between 50° C. and 70° C. and at a pressure between 0.012 and 0.085 MPa, or between 0.012 and 0.03 MPa, and can be followed by controlled cooling down to a temperature between 0° C. and 5° C. The cooling can be accompanied by controlled reduction of the pressure to a pressure of between 0.006 MPa and 0.008 MPa. The cooling can be performed using an internal exchanger and/or by circulation of a heat exchange fluid (in particular water) in a jacket with which is equipped the reactor in which step c) is performed.

The vanillin or the vanillin derivative obtained in the form of a precipitate after step c) can optionally be isolated in step d) of the process in order to improve its purification. Depending on the purity needed, step d) may not be performed in some aspects. Step d) can comprise at least one step of recovering solid vanillin on a filter or spin-dryer, followed by one or more steps of washing with water, preferentially followed by at least one drying step.

The solid vanillin obtained from the precipitation step c) can be recovered on a filter or on a spin-dryer. In order to remove the residual impurities, especially mineral salts including sulfates, one or more washes with an aqueous fluid may be used. The vanillin can then be dried, to be sold in its existing form. It may also be ground and/or recrystallized from water or from a water/alcohol mixture according to a known process. The washing and drying steps performed in the context of the present disclosure can be performed according to standard protocols that are well known to those skilled in the art.

Thus, according to some embodiments, the process may comprise, after step d), a step of recrystallization of the vanillin from water or from an alcohol/water mixture. The vanillin thus obtained can be in the form of white crystals.

In some embodiments, it is possible to further improve the yields by performing one or more additional recycling steps. These additional steps can include recycling various effluents obtained during the process, for example during the extraction, precipitation or washing step. For example, it is possible to recycle the washing waters (e.g., the water recovered after the washing steps) and to use them as process water, e.g., with the water added before and/or the implementation of the evaporation step a) according to the process.

It is also possible to recover some of the vanillin contained in the mother liquors obtained after the precipitation step. This recovery may be performed either by modifying the pH followed by extraction with a solvent, or by reconcentration and precipitation, or by reconcentration and extraction. It is also possible to recover, by washing, some of the vanillin contained in the organic phase obtained after the extraction step.

Other methods of purifying the vanillin obtained from the fermentation process can be used. In some aspects, the present disclosure relates to a process for purifying a stream resulting from a process for producing natural vanillin, the stream comprising natural vanillin and at least vanillyl alcohol, by distillation to enable the separation of the natural vanillin from the vanillyl alcohol. At the end of the process for preparing natural vanillin, a liquid stream (F1) comprising the natural vanillin, the ultra-lights, the lights and the heavies can be obtained. In some embodiments, the stream (F1) comprises: from about 0.5% to about 35% by weight of natural vanillin; from 0.05% to 10% by weight of vanillyl alcohol; from 0.0% to 2% by weight of vanillic acid; from 0.0% to 5% by weight of ferulic acid; from 0.0% to 5% by weight of guaiacol, in particular from 0.01% to 0.6% by weight.

The operating pressure during distillation can be between 2 and 10 mbar, or between 3 and 6 mbar. In the context of the present disclosure, the term “operating pressure” is intended to mean the pressure at the top of the distillation column. Advantageously, the pressure drop in the distillation column may be as small as possible. For example, the pressure drop can be between 1 and 10 mbar, preferably between 2 and 8 mbar.

In the process, one or more distillation columns can be used. The distillation column(s) comprise(s) from 7 to 40 theoretical plates, or from 12 to 30 theoretical plates. Advantageously, the process makes it possible to obtain a natural vanillin having a purity greater than or equal to 96%, or greater than or equal to 98%. The process for purifying natural vanillin by distillation can be carried out in a different manner: by batch distillation or by continuous distillation.

In some embodiments, the process can be carried out by batch distillation. In this process, various unitary distillation operations making it possible to distinctly separate the ultralights, the lights, the vanillin and the heavies. In the batch process, the stream (F1) can be charged to a distillation column. The process makes it possible to recover: in a first fraction, the ultralights; in a second fraction, the lights which may optionally comprise natural vanillin at a low concentration; in a third fraction, the stream comprising the natural vanillin; and in the distillation concentrate, the heavies which may optionally comprise natural vanillin at a low concentration. As used herein, the term “low concentration” means a concentration of at most 10%, or at most 5%, for example of at most 3% by weight relative to the total weight of the fraction. The first, second and third fractions can be recovered by condensation using conventional devices known to those skilled in the art.

Those skilled in the art are capable of setting the distillation parameters, such as in particular the column diameter, the column height and the packing. The following will simply be mentioned. The size (in particular the diameter) of the distillation columns depends on the stream circulating and on the internal pressure. They will therefore be sized mainly according to the flow rate of mixture to be treated. It will be specified that the column can be packed without distinction with plates or with stacked or woven packing, as is fully known to those skilled in the art. Once the facility has been determined, those skilled in the art adjust the operating parameters of the column.

In some aspects, the distillation column comprises from 7 to 30 theoretical plates, preferably from 5 to 15 theoretical plates, for example 7 to 10 theoretical plates. In some aspects, the packing of the column is a woven packing with a surface area between about 400 to 600 m²/m³. This distillation process makes it possible to obtain the natural vanillin with a yield of 90% to 99%, or 90% to 93%.

In some embodiments, the process can be carried out by continuous distillation. For example, the process carried out by continuous distillation can comprise the following steps: a) in a first step, the stream (F1) can be treated in order to remove the ultralights (stream (F11)) that it contains and more particularly the ethyl acetate; the resulting stream comprising the natural vanillin, the lights and the heavies is called stream (F2); b) in a second step, the stream (F2) can be fed into a first distillation column, making it possible to recover, at the top of the distillation, a stream (F12) comprising the lights and which may optionally comprise a low concentration of natural vanillin, and at the bottom of the distillation, a stream (F13) comprising the natural vanillin and the heavies and which may optionally comprise a low concentration of lights; c) the stream (F13) can be continuously fed into a second distillation column, making it possible to recover, at the top of the distillation, a stream (F14) comprising the natural vanillin, and at the bottom of the distillation, a stream (FI5) comprising the heavies and optionally a low concentration of natural vanillin.

According to some embodiments of the topping/tailing scheme, at least one part of the stream (F15) can be mixed with the stream (F14) so as to be able to vary the final composition of a stream of purified natural vanillin. The stream (F12) at the top of the distillation in step b) can comprise the lights, and in particular benzoic acid and guaiacol and optionally a low concentration of natural vanillin.

The distillation column of step b) can comprises from 20 to 35 theoretical plates, or from 23 to 30 theoretical plates. The distillation column of step b) can comprises from 8 to 15 theoretical plates for the concentration section and from 12 to 20 theoretical plates for the depletion section, or from 10 to 13 theoretical plates for the concentration section and from 13 to 17 theoretical plates for the depletion section. In the column of step b), the operating pressure can be between 2 mbar and 10 mbar, or between 3 mbar and 6 mbar. The pressure drop can be from 1 to 10 mbar, or from 1 to 8 mbar. During step b), the reflux ratio can be between 10 and 30, or between 15 and 25. The reflux ratio is defined as the ratio of the flow rate of material reinjected from the top of the column to the inside of the column (e.g. the reflux flow rate) to the flow rate actually exiting at the outlet of the top of the column. The stream (F2) feed point is selected in a manner known to those skilled in the art; it is in particular selected in such a way as to avoid back-mixing. Preferably, the feed point is at the top of the depletion section. The ratio of the distillation flow rate (flow rate of the stream (F12)) to the feed flow rate (flow rate of the stream (F2)) can be from 0.05 to 0.5. The reflux flow rate can be from 1.4 to 3 times the feed flow rate; for example, it can be twice the feed flow rate. Preferably, during step b), the temperature at the bottom of the column can be between 100° C. and 160° C., or between 120° C. and 150° C., and during step b), the temperature at the top of the column can be between 80° C. and 120° C., or between 90° C. and 110° C. The residence time of the vanillin at the bottom of the column of step b) can be between 30 minutes and 5 hours, for example 3 hours.

The distillation can be initiated by adjusting the flow rate of stream (F12) so as to maintain an approximate temperature of preferably from 100° C. to 160° C. The temperature difference between the top (vapor temperature) and the bottom (temperature of the liquid) can be between 35° C. and 45° C. The stream (F13) at the bottom of the column at the end of step b) can be sent to the distillation column of step c), also called tailing column. In some aspects, the column of step c) comprises from 10 to 30 theoretical plates, or from 15 to 25 theoretical plates. The distillation column of step c) comprises from 4 to 14 theoretical plates for the depletion section and from 6 to 16 theoretical plates for the concentration section. In the column of step c), the operating pressure can be between 2 and 10 mbar, or between 3 and 6 mbar, for example 4 mbar. The pressure drop can be preferably from 1 to 10 mbar, preferably from 1 to 8 mbar, for example, mbar. During step c), the reflux ratio can be between 2 and 10, or between 2.5 and 6, for example, 3. The ratio of the distillation flow rate (flow rate of the stream (F14)) to the feed flow rate (flow rate of the stream (F2) can be from 0.5 to 0.95. The reflux flow rate can be from 1.4 to 3 times the feed flow rate; for example, it can be twice the feed flow rate. In some aspects, during step c), the temperature at the bottom of the column can be between 160° C. and 200° C., for example between 170° C. and 190° C. In some aspects, during step c), the temperature at the top of the column can be between 100° C. and 160° C., or between 110° C. and 140° C.

The residence time of the vanillin at the bottom of the column of step c) can be between 15 minutes and 15 hours, between 30 minutes and 15 hours, or between 7 and 15 hours, in order to reduce this residence time to between 15 minutes and 8 hours and to limit the degradation of the natural vanillin, which is heat-sensitive, it may be advantageous to add a deadweight at the bottom of the column. Deadweights are well known to those skilled in the art, for example they may comprise glass beads.

In order to perform the distillation of step c), heat can be supplied to the bottom of the column in particular by a falling-film or scraped-film boiler, by a shell-and-tube heat exchanger heated with steam or by a heat-transfer fluid, by means of heating coils fed with steam or by a heat-transfer fluid or by any other equivalent device, preferably by a heat-transfer fluid. Preferably, the energy for the distillation is supplied by a forced-circulation shell-and-tube device or a scraped exchanger.

In some embodiments, the process makes it possible to obtain a very pure natural vanillin, in particular having a purity greater than or equal to 99% and preferably containing less than 1000 ppm of vanillyl alcohol. This embodiment can be obtained by adjusting the distillation parameters, in particular the ratio of the distillation flow rate (F14) to the feed flow rate (F2) is then preferably between 0.67 and 0.734.

The present disclosure is also directed to a purification process that involves the use of hot water to purify vanillin product. In some embodiments, the vanillin-containing fermentation product can be extracted by organic solvents including any of those described herein (e.g., ethyl acetate, etc.). A volume ratio of fermentation product to organic solvent can range from 0.5:1 to 5:1. The organic phase can be separated from aqueous layer via separatory funnel. The collected organic phase can be concentrated to dryness via distillation. The obtained dried product can be a yellow to brown oil liquid, which contains vanillin, aromatic impurities like guaiacol, salts and unknown metabolite. Certain impurities like salts can be precipitated via washing with organic solvents. Thus, the yellow to brown oil liquid is first washed with organic solvents (mass ratio of oil liquid to solvent ranges from 1:1 to 1:10) like acetone or ethanol etc. to crash out of solution solids like salts. The organic solvent can be filtered and concentrated to yield a new yellow to brown oil liquid. It is found that certain impurities have low solubility in boiling water. Thus, a hot water extraction step is used to separate those impurities. The oil liquid product is introduced with 1 to 10 mass equivalent amount of water and the mixture is heated to 45° C. to 95° C. Vanillin can be extracted into hot water while insoluble impurities are filtered off when hot. The vanillin-containing filtrate can be cooled down to room temperature and a yellowish cloudy solution is obtained. The solution can be extracted with equal volume of an organic solvent for 1-3 times to extracted vanillin into the organic phase. The organic phase can be dried via distillation to dryness and a light yellowish solid can be achieved with vanillin purity to be 80-90%. Washing the solid with water can increase the vanillin purity to >95%.

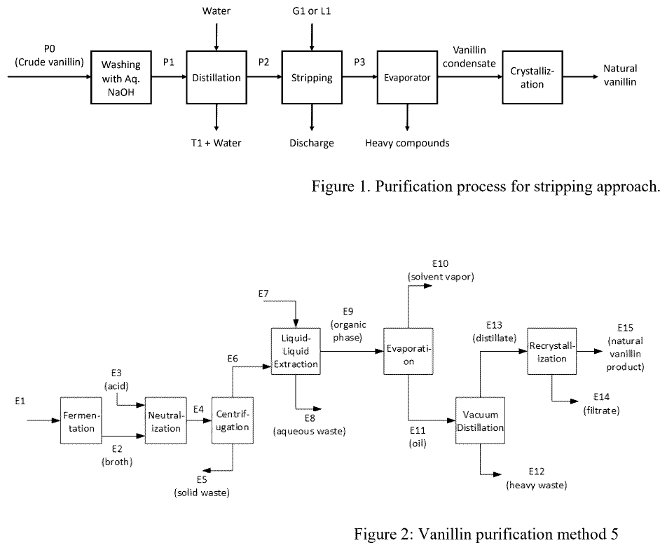

The present disclosure is also directed to a purification process that involves the stripping to produce a vanillin product. As shown in FIG. 1, some aspects relate to a process for the purification of natural vanillin comprising at least one step (b) of stripping a liquid stream P2 comprising natural vanillin with an entraining gas G1 and/or a vaporized liquid L1.

The liquid stream P2 may result from a process for the production of natural vanillin, including any of those disclosed herein. In this stream P2, the natural vanillin exists in the form of non-salified vanillin. In the liquid stream P2 of natural vanillin, the concentration by weight of vanillin can be greater than or equal to 10%, greater than or equal to 30%, greater than or equal to 50%, or greater than or equal to 70%, with respect to the total weight of the liquid stream.

A process for the production of natural vanillin denotes in this instance a biotechnological process comprising the culturing of a microorganism capable of making possible the bioconversion of a fermentation substrate into vanillin. In some aspects, it is a process for the fermentation of ferulic acid.

Apart from the vanillin, the stream P2 may contain impurities, in particular impurities formed during the production by enzymatic conversion or by fermentation, typically vanillyl alcohol, vanillic acid, dimers and trimers of vanillin (that is to say, compounds exhibiting a backbone having respectively two or three phenyl groups, the dimers advantageously being chosen from diphenylmethanes). When a fermentation of ferulic acid is concerned, the typical impurities may in addition be chosen from ferulic acid, guaiacol and guaiacol derivatives. Finally, the stream P2 may contain traces of stabilizer. In addition, the stream P2 may comprise a solvent or a mixture of several solvents, such as a food-grade solvent or water. However, the concentration by weight of solvent in the liquid stream P2 can be less than or equal to 90%, less than or equal to 70%, less than or equal to 50%, or less than or equal to 30%, with respect to the total weight of the liquid stream.

The process according to the disclosure may be carried out according to a continuous operation or according to a batchwise operation. In some aspects, the stripping can comprise a step of entraining via an entraining gas or a vaporized liquid. This is because impurities are entrained by the gas or vaporized liquid, so as to improve the quality of the vanillin. In particular in the context of the present disclosure, the stripping step makes it possible advantageously to remove certain impurities present in the stream of natural vanillin. Such a stripping step may be carried out under mild conditions, in particular by injection of a gaseous fluid or of a liquid and/or placing under vacuum the chamber where the process according to the disclosure is carried out.

In the context of the present disclosure, the entraining gas G1 or the vaporized liquid L1 is chosen from the group consisting of water, steam, alkyl acetates, alcohols, inert gases chosen from N₂, CO₂, He, Ar, depleted air and their mixtures. Preferably, the entraining gas G1 or the vaporized liquid L1 is water or steam. The use of a mixture of several entraining gases G1 and/or of several vaporized liquids L1 may be envisaged.

Step (b) may be carried out at a temperature of greater than or equal to 20° C., greater than or equal to 30° C., greater than or equal to 40° C., or greater than or equal to 50° C. Step (b) may be carried out at a temperature of less than or equal to 140° C., less than or equal to 120° C., less than or equal to 100° C., or at about 95° C. According to some embodiments, step (b) can be carried out under decreasing vacuum ranging from 400 mbar to 25 mbar. According to some embodiments, the stripping step can be carried out with an entraining gas G1, for example with steam.

This embodiment may be carried out continuously in a stripping column. Typically, the liquid stream P2 can be introduced into the stripping column via the top, while the entraining gas G1 can be introduced via the bottom. During their contact, the entraining gas becomes charged with impurities present in the liquid stream and be extracted via the top of the column, while the purified liquid stream can be recovered at the column bottom.

In some aspects, the stripping step can be carried out with a vaporized liquid L1, preferably with water. This embodiment may be implemented continuously or batchwise in a reactor. Typically, the liquid stream P2 and the liquid L1 can be mixed in the reactor. Then, by reducing the pressure and/or increasing the temperature, the liquid L1 vaporizes. The vaporized liquid L1 can then be extracted from the reactor, entraining with it impurities present in the liquid stream. The liquid stream, thus purified, may then be recovered in the reactor. Preferably, the stripping step (b) can be carried out under an inert atmosphere and more preferably under N₂.

According to a preferred embodiment, the process for the purification of natural vanillin according to the disclosure may additionally comprise a preliminary step (a) of preparation of the liquid stream P2 of natural vanillin by evaporating, optionally in the presence of water, the solvent T1 of a stream P1 originating from the production of natural vanillin. This is because, during the production of natural vanillin, it is typical to recover a liquid stream comprising natural vanillin, impurities and a large amount of solvent, typically a food-grade solvent, such as, for example, ethyl acetate. This stream is typically denoted “crude vanillin solution”.

The process according to the disclosure may comprise a step of making available a liquid stream P1 resulting from a process for the production of natural vanillin comprising natural vanillin in a solvent T1. Mention may be made, among the solvents T1, for example, of the organic solvents authorized by the regulations, such as MEK (methyl ethyl ketone), alcohols (ethanol, butanol, and the like), alkyl acetates (ethyl acetate, propyl acetate, isopropyl acetate, and the like), MIBK (methyl isobutyl ketone) and cyclohexane. The solvent T1 may also be water. The solvent T1 may also be a mixture of organic solvents, in particular a mixture of the organic solvents mentioned above, or a mixture of water and of an organic solvent.

According to one embodiment, the liquid stream P1 may additionally comprise a stabilizer for the fermentation medium. The bacteriostatic agents (or biocides) which may be employed as stabilizers are well known to a person skilled in the art, for example sorbic acid, benzoic acid, acetic acid and their salts. The stabilizer for natural vanillin in the liquid stream P1 can be generally present in an amount of less than or equal to 20% by weight, or less than or equal to 10% by weight. The liquid stream P2 may consequently contain traces of the stabilizers. However, the stream F1 may also be devoid of stabilizer, in particular when the stabilization of the fermentation medium has been carried out in another way. For example, the liquid stream P1 may have been stabilized by heat treatment. This heat treatment makes it possible to halt the action of the microorganisms. The temperature of the heat treatment can generally be greater than or equal to 35° C. and generally less than or equal to 110° C., for example between 50° C. and 110° C.

According to a specific embodiment of the present disclosure, the process additionally comprises a preliminary step, before step (a) or before step (b), of washing the stream resulting from the process for the production of natural vanillin. This washing may be carried out using an aqueous solution, in order to remove the acidic impurities. In general, this washing may be carried out with a basic solution, preferably a sodium hydroxide solution.

Preferably, in the stream P1, the concentration by weight of the vanillin can be from 0.5% to 60%, from 5% to 40%, or from 10% to 35%, with respect to the total weight of the stream.

The optional step (a) according to the present disclosure can include removing the solvent T1 in order to obtain a stream P2 of vanillin in which the concentration by weight of vanillin is greater than or equal to 10%, greater than or equal to 30%, greater than or equal to 50%, or greater than or equal to 70%, with respect to the total weight of the stream. The residual content of solvent T1 can be less than or equal to 90%, less than or equal to 70%, less than or equal to 50%, or less than or equal to 30%, with respect to the total weight of the liquid stream.

In accordance with the process according to the disclosure, the evaporation step (a) may optionally be carried out in the presence of water, which is added to the stream P1 before and/or during the implementation of the evaporation step.

In a preferred embodiment, in the context of step (a), the solvent Tl for the crude vanillin can be removed by evaporation, for example by distillation or by means of an evaporator, in the presence of water. The water and the solvent T1 may form an azeotropic mixture. In the case of an evaporation by distillation, the solvent SI may be distilled at atmospheric pressure or under vacuum or alternatively at atmospheric pressure and then under vacuum. The water may be added in one or more goes to the stream P1. It can be preferable to use food-grade water (for example municipal water). It is also possible to use recycled food-grade water originating from the process according to the present disclosure.

Preferably, the evaporation step (a) can be carried out at a temperature of between 60° C. and 140° C., or between 80° C. and 100° C.

After step (b), the process for the purification of natural vanillin according to the disclosure may additionally comprise a step (c) of removal of the less volatile compounds than vanillin. This step may advantageously be carried out in a vacuum film evaporator or in a thin film evaporator.

Additional methods for purifying vanillin are also disclosed herein. As shown in FIG. 2, the present processes and systems thus relate to a process for purifying vanillin, starting with a fermentation broth E2, containing vanillin in water, containing impurities, comprising the following steps: a) a step of neutralization and centrifugation of fermentation broth to remove cellular biomass/debris and particulate matter; b) a step of extraction (e.g., liquid-liquid extraction, etc.) by placing the aqueous solution obtained after step a) in contact with a solvent E7, at a pH greater than about 4 and less than about 10, to obtain an organic phase containing vanillin and impurities, and an aqueous phase containing water-soluble impurities, salts, and residual solvent S2; c) a step of distillation (e.g., which can be carried out under vacuum), at a pressure of between 2 and 20 mbar, of the vanillin contained in the concentrated oil obtained from the evaporation of the organic phase obtained after step b), and d) a step of multi-step recrystallization of the vanillin.

FIG. 2 illustrates a vanillin purification method 5 as described. In summary, ferulic acid starting material (E1) is fed to fermentation yielding fermentation broth (E2) containing vanillin. The fermentation can be carried out using any of the processes described herein including the use of Amycolatopsis tucumanensis (DSM 45259, LGM 24814) as described herein. The stream E2 can be neutralized with acid (E3) and the neutral or acidic product mixture (E4) can be centrifuged, yielding solid waste (E5) and an aqueous product stream containing vanillin (E6). Aqueous vanillin stream E6 can be mixed with an organic solvent (E7), following phase separation aqueous waste (E8) can be separated from the vanillin containing organic phase (E9). Stream E9 undergoes evaporation to remove solvent vapor (E9) and produce a concentrated vanillin oil (E12) which undergoes distillation where vanillin can be concentrated in the distillate (E13) and recrystallized to yield a natural vanillin product of high purity (E15).

In some embodiments, the process of the present disclosure comprises a step a) that comprises neutralizing the fermentation broth, followed by removing particulate and cellular solids in the fermentation broth, for example, using centrifugation or other suitable techniques. In accordance with the process, the neutralization step can be performed using organic or inorganic acids, including but not limited to hydrochloric, sulfuric, or other suitable mineral acids. Exemplary, nonlimiting acids can include, but are not limited to 36% hydrochloric acid or 98% sulfuric acid. Preferably, the pH of the neutralized fermentation broth is between 3 and 10, especially between 5 and 8.

After the neutralization of the fermentation broth, the solids can be removed by centrifugation. Solids may also be removed by membrane filtration. In some aspects, the neutralization and centrifugation step a) can be performed at a temperature between 20° C. and 40° C.

After step a), the obtained aqueous solution of vanillin or of a vanillin derivative is subjected to an extraction step b) under particular pH conditions. While any suitable extraction step can be used (e.g., solid adsorbent extraction), in some aspects, the extraction can use liquid-liquid extraction. The solvent used for this extraction step can be referred to herein below as solvent E7.

Step b) can be performed at a controlled pH so as to separate certain impurities from vanillin by pKa difference. In particular, step b) can be performed so as to extract the impurities formed by species with a lower pKa than that of Vanillin. The extraction according to step b) may be total or partial. In accordance with step b) of the process, the pH can be chosen so as to obtain a high yield of vanillin. Thus, the pH can be greater than 6 and less than 10.

During this step of extraction at controlled pH, the protonated species can be extracted by Solvent E7 and the organic layer predominantly comprises solvent E7. The vanillin can be extracted into the organic phase. For the rest of the process, the organic phase thus obtained can be used, e.g., the steps subsequent to the extraction step can be performed using the organic phase containing the vanillin.

The solvent E7 can include any of those organic solvents disclosed herein. According to some embodiments, the extraction solvent E7 can be different from solvent E2 present in the initial vanillin solution. In accordance with the disclosed process, solvent E2 and solvent E7 can be chosen from the solvents permitted by the regulations in force according to directive 2009/32/EC and directive 2010/59/UE concerning extraction solvents used in the manufacture of foodstuffs and ingredients thereof. In accordance with step b) of the process, the extraction solvent E7 has no or very low to moderate solubility in water. More precisely, the maximum weight content of the solvent E7 in water can be equal to 70 g/L. In some aspects, the solvent E7 is an organic solvent. Preferably, solvent E7 can be a solvent with low or very low solubility in water, e.g., its maximum weight content in water can be less than or equal to 50 g/L, for example, less than or equal to 20 g/L. Solvent E7 may also be a water-insoluble solvent. The extraction solvent E7 advantageously has a boiling point less than 200° C., or less than 150° C. Among the solvents E7 employed for the implementation of step b) of the process, examples that may be mentioned include dichloromethane, cyclohexane, ethyl acetate, propyl acetate, isopropyl acetate, n-butyl acetate, isoamyl acetate, methyl isobutyl ketone (MIBK), butanol, Rhodiasolv RPDE (mixture of dimethyl adipate, dimethyl Succinate and dimethyl glutarate), or a mixture of these solvents.

According to an advantageous embodiment, solvent E7 can be chosen from food grade solvents. In some aspects, solvent E7 can be chosen from the group consisting of ethyl acetate, propyl acetate, isopropyl acetate, n-butyl acetate, isoamyl acetate and methyl isobutyl ketone (MIBK), and mixtures thereof. In some aspects, solvent E7 can be isopropyl acetate.

In order to obtain the particular pH conditions described above, according to one embodiment, step b) of the process comprises the addition of a base, the base can be either a weak base or a strong base. In some aspects, the base can be chosen from mineral bases, and more particularly water-soluble mineral bases. In particular, the base can be chosen from the group consisting of alkali metal hydroxides, alkaline-earth metal hydroxides, alkali metal bicarbonates, alkaline-earth metal bicarbonates, alkali metal hydrogen carbonates, alkaline-earth metal hydrogen carbonates, alkali metal phosphates, alkaline-earth metal phosphates, alkali metal hydrogen phosphates and alkaline-earth metal hydrogen phosphates, or any mixtures thereof. In some embodiments, the base can be chosen from the following mineral bases: NaOH, KOH and Na₂CO₃, or combinations thereof. For example, NaOH or KOH can be used as base, and more particularly NaOH. This base addition step makes it possible to adjust the pH for the extraction step to a pH value greater than about 6 and less than about 10.

In accordance with step b) of the process, the addition of the base to the vanillin solution can be performed before the addition of solvent E7 or after the addition of solvent E7. According to some embodiments, the base can be added rapidly to the vanillin solution, preferably in a single portion. According to one embodiment of the process, the base can be added to the vanillin solution, optionally containing solvent E7, at a temperature from 15° C. to 60° C. or between 3° and 50° C. According to one embodiment of the process, the base advantageously used for performing the step b) is diluted in water to a concentration from 5% to 30% by weight relative to the weight of the aqueous solution in which the base is diluted.

The liquid-liquid extraction step is preferably performed at atmospheric pressure and at a temperature between 15° C. and 40° C. and preferentially between 20° C. and 30° C. Preferably, the extraction step is preceded by a cooling step. According to an embodiment, the base is added to the vanillin solution obtained after the step a) at a temperature from 15° C. to 60° C., or from 20° C. to 50° C., and the temperature can be then lowered to a temperature between 15° C. and 40° C. or between 20° C. and 30° C. before adding solvent E7 for the extraction.

After step b), an organic phase E9 and an aqueous phase E8 can be obtained. The organic phase E9 contains the vanillin, and some of the impurities, especially the guaiacol, vanillyl alcohol and dimers, and solvent E7, whereas the aqueous phase contains the rest of the impurities, for example ferulic acid, vanillic acid, benzoic acid and/or the dimers comprising a ferulic unit, and also residual solvent E7. The solvent E7 present in the aqueous phase, preferentially freed of impurities, is advantageously recycled upstream of the step b).

According to a batchwise procedure of the process, the organic phase can be then preferentially allowed to separate out by settling to recover the aqueous phase.

According to a continuous procedure of the process, the liquid/liquid extraction step can be performed by using a series of decanting mixers or at least one stirred, pulsed or packed liquid/liquid extraction column. It may also be performed continuously by using a static mixer and then a centrifugal separator for continuously separating the organic and aqueous phases.

After step b), the vanillin or the vanillin derivative is present in organic phase. This solution contains a large proportion of the impurities present in the crude vanillin, in particular the species whose pKa is more than that of vanillin. These species are, in particular, guaiacol, vanillyl alcohol, other unidentified impurities from the fermentation broth, or other impurities originating from use of low purity ferulic acid feed in the fermentation of ferulic acid to vanillin.

After isolating the organic phase in step b), the solvent can be removed by evaporation in step c). Evaporation of the organic solvent can be performed under vacuum. Vacuum evaporation can be performed at temperatures between 40° C. and 60° C., and at pressures between 100 and 200 mbar. The concentrate obtained from evaporating the organic phase of step b) is an orange or brown oil. At this stage of the process for preparing natural vanillin, comprising the natural vanillin, the ultra-lights, the lights and the heavies is obtained. The concentrated oil comprises: from 5% to 35% by weight of natural vanillin; from 0.00% to 10% by weight of vanillyl alcohol; from 0.0% to 2% by weight of vanillic acid; from 0.0% to 2% by weight of ferulic acid; from 0.0% to 2% by weight of guaiacol, in particular from 0.01% to 0.6% by weight. This concentrated oil can be the feed for vacuum distillation. In some aspects, the operating pressure during distillation can be between 2 and 20 mbar, or between 3 and 6 mbar. In some aspect, the pressure drop in the distillation column can be between 1 and 10 mbar, or between 2 and 8 mbar.

In the process, a short-path distillation column can sometimes be used to increase product recovery.

Advantageously, the vacuum distillation makes it possible to obtain a natural vanillin having a purity greater than or equal to 70%, or greater than or equal to 80%.

The process for purifying natural vanillin by distillation can also be carried out in a different manner such as by batch distillation or by continuous distillation. In one embodiment, the process can be carried out by batch distillation. The process thus comprises various unitary distillation operations making it possible to distinctly separate the ultralights, the lights, the vanillin and the heavies.

In the batch process, the concentrated oil can be charged to a distillation column. The process makes it possible to recover: in a first fraction, the ultralights; in a second fraction, the lights which may optionally comprise natural vanillin at a low concentration; in a third fraction, the stream comprising the natural vanillin; and in the distillation concentrate, the heavies which may optionally comprise natural vanillin at a low concentration.

The first, second and third fractions can advantageously be recovered by condensation using conventional devices known to those skilled in the art.

Those skilled in the art are capable of setting the distillation parameters, such as in particular the column diameter, the column height and the packing. The following will simply be mentioned. The size (in particular the diameter) of the distillation columns depends on the stream circulating and on the internal pressure. They will therefore be sized mainly according to the flow rate of mixture to be treated. It will be specified that the column can be packed without distinction, with plates, or with stacked or woven packing, as is fully known to those skilled in the art. Once the facility has been determined, those skilled in the art adjust the operating parameters of the column.

This distillation process makes it possible to obtain the natural vanillin with a yield of 90% to 99%, or of 94% to 98%.

In some embodiments, the process can be carried out by continuous distillation. In one particular embodiment, the process carried out by continuous distillation comprises the following steps: a) in a first step, the concentrated oil (F1) can be treated in order to remove the ultralights (stream (F11)) that it contains and more particularly the ethyl acetate; the resulting stream comprising the natural vanillin, the lights and the heavies is called stream (F2); b) in a second step, the stream (F2) can be fed into a first distillation column, making it possible to recover, at the top of the distillation, a stream (F12) comprising the lights and which may optionally comprise a low concentration of natural vanillin, and at the bottom of the distillation, a stream (F13) comprising the natural vanillin and the heavies and which may optionally comprise a low concentration of lights; c) the stream (F13) can be continuously fed into a second distillation column, making it possible to recover, at the top of the distillation, a stream (F14) comprising the natural vanillin, and at the bottom of the distillation, a stream (FI5) comprising the heavies and optionally a low concentration of natural vanillin.

Preferably, the stream (F12) at the top of the distillation in step b) comprises the lights, and in particular benzoic acid and guaiacol and optionally a low concentration of natural vanillin. In the column of step b), the operating pressure can be between 2 mbar and 10 mbar, or between 3 mbar and 6 mbar. The pressure drop can be from 1 to 10 mbar or from 1 to 8 mbar.

The stream (F2) feed point can be selected in a manner known to those skilled in the art. It is in particular selected in such a way as to avoid back-mixing. Preferably, the feed point can be at the top of the depletion section.

During step b), the temperature at the bottom of the column can be between 100° C. and 160° C., or between 120° C. and 150° C. During step b), the temperature at the top of the column can be between 80° C. and 120° C., or between 90° C. and 110° C. The residence time of the vanillin at the bottom of the column of step b) can be between 30 minutes and 5 hours. The distillation can be carried out by adjusting the flow rate of stream (F12) so as to maintain an approximate temperature of preferably from 100° C. to 160° C. The temperature difference between the top (vapor temperature) and the bottom (temperature of the liquid) can be preferably between 35° C. and 45° C.

The stream (F13) at the bottom of the column at the end of step b) is sent to the distillation column of step c), also called tailing column.

In the column of step c), the operating pressure can be preferably between 2 and 10 mbar, or between 3 and 6 mbar, for example 4 mbar. The pressure drop can be from 1 to 10 mbar, or from 1 to 8 mbar, for example, it is 4 mbar. The ratio of the distillation flow rate (flow rate of the stream (F14)) to the feed flow rate (flow rate of the stream (F2) can be from 0.5 to 0.95. During step c), the temperature at the bottom of the column can be between 160° C. and 200° C., in particular between 170° C. and 190° C. Preferably, during step c), the temperature at the top of the column is between 100° C. and 160° C., preferably between 90° C. and 130° C.

The residence time of the vanillin at the bottom of the column of step c) can be between 15 minutes and 15 hours, preferably between 30 minutes and 15 hours, more preferably between 7 and 15 hours, in order to reduce this residence time to between 15 minutes and 8 hours and to limit the degradation of the natural vanillin, which is heat-sensitive, it may be advantageous to add a deadweight at the bottom of the column.

In order to perform the distillation of step c), heat can be supplied to the bottom of the column in particular by a falling-film or scraped-film boiler, by a shell-and-tube heat exchanger heated with steam or by a heat-transfer fluid, by means of heating coils fed with steam or by a heat-transfer fluid or by any other equivalent device, such as by a heat-transfer fluid. In some aspects, the energy for the distillation is supplied by a forced-circulation shell-and-tube device or a scraped exchanger.

In some embodiments, the process makes it possible to obtain a very pure natural vanillin, in particular having a purity greater than or equal to 99% and preferably containing less than 1000 ppm of vanillyl alcohol. This embodiment can be obtained by adjusting the distillation parameters.

In some embodiments, the isolation of the vanillin can be carried out by recrystallization with montmorillonite decolorization. The vanillin or the vanillin derivative obtained in the form of a liquid oil c) can be isolated in step d) of the process in order to improve its purity. Step d) advantageously comprises of one or more recrystallizations in water, one of which utilizes a decoloring agent including but not limited to montmorillonite or activated carbon to remove color, preferentially followed by at least one drying step. The vanillin obtained from step c) may be recrystallized from water or from a water/alcohol mixture. Thus, according to one embodiment, the process may comprise, after step c), a step of recrystallization of the vanillin from water or from an alcohol/water mixture. The vanillin thus obtained can be in the form of lightly colored or white crystals or solids.

In some embodiments, it is possible to further improve the yields by performing one or more additional recycling steps. These additional steps comprise recycling various effluents obtained during the process, for example during the extraction, precipitation or washing step. For example, it is possible to recycle the washing waters (e.g., the water recovered after the washing steps) and adding them to the solvent extraction step b) where the vanillin is recovered in the organic phase. It is also possible to recover some of the vanillin contained in the mother liquors obtained after the precipitation step. This recovery may be performed either by modifying the pH followed by extraction with a solvent, or by reconcentration and precipitation, or by reconcentration and solvent extraction.

In the case of a yellow-colored vanillin crystal product, the vanillin can be decolorized by performing a recrystallization where the vanillin is dissolved in water or a water/alcohol mixture, and montmorillonite clay is added in proportion to the amount of vanillin in solution, between 0.1 and 0.5 After adding the montmorillonite clay, the solution can be filtered through a 0.45 micron filter, or with the use of filter aid, such as celite, to obtain a clear, decolorized solution. The filtrate is then concentrated and recrystallized. In some aspects, the resulting natural vanillin has a purity greater than or equal to 96% that is in the form of an amorphous solid of which the color, in ethanolic solution at 10% by weight, is less than or equal to 100 Hazen. The resulting product can be further processes spalling, pelletizing or prilling to develop the final product.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

Note on source of strain. Amycolatopsis tucumanensis LGM 24814 used for the processes and experiments described herein was purchased from Belgian Coordinated Collections of Microorganisms (BSSM) listed under the BCCM catalog number LGM 24814.

### Example 1

### Preparation of Preliminary Culture

A 250 mL baffled erlenmeyer flask was filled with 100 mL of sterilized medium. The medium consisted of 4 g/L yeast extract, 0.2 g/L NaCl, 0.05 g/L CaCl₂)·2H₂O, 0.2 g/L MgSO₄·7H₂O, 4 g/L Na₂HPO₄, 1 g/L KH₂PO₄, 10 g/L Dextrose in water. The flask was inoculated with 200 microliters of a frozen glycerol culture of Amycolatopsis tucumanensis (LGM 24814). The culture was incubated on a rotary shaking machine at 28° C. and 250 rpm. After 96 hours this culture was used to inoculate the production medium.

### Example 2

Production of Vanillin in Shaker Flasks with High Purity Synthetic Ferulic Acid.

Three 250 mL baffled erlenmeyer flasks were each filled with 30 mL of sterile medium (4 g/L yeast extract, 0.2 g/L NaCl, 0.05 g/L CaCl₂·2H₂O, 0.2 g/L MgSO₄· 7H₂O, 4 g/L Na₂HPO₄, 1 g/L KH₂PO₄, 10 g/L Dextrose in water). The flasks were each inoculated with 2 mL of a culture of Amycolatopsis tucumanensis (LGM 24814) from Example 1. The cultures were incubated in a rotary shaker at 28° C. and 250 rpm. 2 mL of sterile filtered 100 g/L ferulic acid (Thermo scientific, 99% pure) were added to each flask 24 hours after inoculation. A small amount of culture media of each sample was removed at 15.5, 23.5, and 42 hours after addition of ferulic acid and analyzed by high performance liquid chromatography (HPLC). HPLC analysis of the three cultures 23.5 hours after ferulic acid addition showed a molar yield of 62.2%+/−4.6% vanillin (molar yield defined as moles vanillin divided by starting moles ferulic acid×100).

### Example 3

Production of vanillin in shaker flasks with low purity natural ferulic acid where the ferulic acid purity is less than 30% by weight.

Three 250 mL baffled erlenmeyer flasks were each filled with 30 mL of sterile medium (4 g/L yeast extract, 0.2 g/L NaCl, 0.05 g/L CaCl₂)·2H₂O, 0.2 g/L MgSO₄·7H₂O, 4 g/L Na₂HPO₄, 1 g/L KH₂PO₄, 10 g/L Dextrose in water). The flasks were each inoculated with 2 mL of a culture of Amycolatopsis tucumanensis (LGM 24814) from Example 1. The cultures were incubated in a rotary shaker at 28° C. and 250 rpm. 2 mL of sterile filtered 100 g/L natural ferulic acid, which was prepared by dissolving natural ferulic acid with a purity of less than 30% by weight, was added to each flask 24 hours after inoculation. A small amount of culture media of each sample was removed at 15.5, 23.5, and 42 hours after addition of ferulic acid and analyzed by HPLC. HPLC analysis of the three cultures 23.5 hours after ferulic acid addition showed a molar yield of 49%+/−13.5% vanillin (molar yield defined as moles vanillin divided by starting moles ferulic acid×100).

### Example 4

Production of Vanillin in Shaker Flasks with Vanillin Absorbing Resin.

Two 2 L baffled erlenmeyer flasks (Flasks A and B) were each filled with 250 mL of sterile medium (4 g/L yeast extract, 0.2 g/L NaCl, 0.05 g/L CaCl₂)·2H₂O, 0.2 g/L MgSO₄· 7H₂O, 4 g/L Na₂HPO₄, 1 g/L KH₂PO₄, 10 g/L Dextrose in water). The flasks were each inoculated with 16 mL of a culture of Amycolatopsis tucumanensis (LGM 24814) from Example 1. The cultures were incubated in a rotary shaker at 28° C. and 250 rpm. 17 mL of sterile filtered 100 g/L ferulic acid (Thermo scientific, 99% pure) were added to each flask 24 hours after inoculation. Immediately following addition of ferulic acid to the cultures, 35 g of sterile and pre-conditioned Amberlite XAD-16N resin was added to the cultures and the cultures were incubated in a rotary shaker at 28° C. and 250 rpm. After 24 hours one of the cultures (Flask A) was removed from the incubator and filtered using a buchner funnel to separate the liquid medium from the solid resin. The liquid media was analyzed by HPLC and found to contain a molar yield of 1.2% vanillin and 18.7% unreacted ferulic acid (molar yield defined as moles product divided by starting moles ferulic acid×100). The solid resin from Flask A was washed with 150 mL ethanol twice and filtered to separate the ethanol from the solid resin. The combined 300 mL ethanol wash was found to contain a molar yield of 31.7% vanillin and 26.1% unreacted ferulic acid. The total molar yield of vanillin from Flask A was 32.9% with 44.8% unreacted ferulic acid.

After 42 hours the second culture (Flask B) was removed from the incubator and filtered using a buchner funnel to separate the liquid medium from the solid resin. The liquid media was analyzed by HPLC and found to contain a molar yield of 3.7% vanillin and 3.7% unreacted ferulic acid (molar yield defined as moles product divided by starting moles ferulic acid×100). The solid resin from Flask B was washed with 150 mL ethanol twice and filtered to separate the ethanol from the solid resin. The combined 300 mL ethanol wash was found to contain a molar yield of 63.8% vanillin and 4.3% unreacted ferulic acid. The total molar yield of vanillin from Flask B was 67.5% with 8.0% unreacted ferulic acid.

### Example 5

Production of Vanillin in Shaker Flasks with Vanillin Absorbing Resin Using Natural Ferulic Acid where the Ferulic Acid Purity is 85%

A 2 L baffled Erlenmeyer flask was filled with 250 mL of sterile medium (4 g/L yeast extract, 0.2 g/L NaCl, 0.05 g/L CaCl₂)·2H₂O, 0.2 g/L MgSO₄· 7H₂O, 4 g/L Na₂HPO₄, 1 g/L KH₂PO₄, 10 g/L Dextrose in water). The flask was inoculated with 16 mL of a culture of Amycolatopsis tucumanensis (LGM 24814) from Example 1. The culture was incubated in a rotary shaker at 28° C. and 250 rpm. 17 mL of sterile filtered 100 g/L ferulic acid (natural ferulic acid, 85% pure) were added to the culture 24 hours after inoculation. Immediately following addition of ferulic acid to the culture, 35 g of sterile and pre-conditioned Amberlite XAD-16N resin was added to the culture and the culture was incubated in a rotary shaker at 28° C. and 250 rpm. After 24 hours the culture was removed from the incubator and filtered using a Büchner funnel to separate the liquid medium from the solid resin. The liquid media was analyzed by HPLC and found to contain a molar yield of 2.3% vanillin and 15.9% unreacted ferulic acid (molar yield defined as moles product divided by starting moles ferulic acid×100). The solid resin was washed with 150 mL ethanol twice and filtered to separate the ethanol from the solid resin. The combined 300 mL ethanol wash was found to contain a molar yield of 43.5% vanillin and 15.5% unreacted ferulic acid. The total molar yield of vanillin was 45.8% with 31.4% unreacted ferulic acid.

### Example 6

In a prophetic example, examples 1-5 were replicated using an incubation temperature of 30-65° C.

### Example 7 (Liquid-Liquid Extraction Method)

In a prophetic example, 1000 g of crude vanillin solution produced according to Example 3 was extracted with 1000 mL of ethyl acetate twice. The ethyl acetate layer was separated by a separatory funnel. The ethyl acetate fraction was fully evaporated using a rotavapor at 65° C. under vacuum (0.5-0.75 bar). The dried product was a brown liquid oil.

200 g of water was added to the brown liquid oil, followed by the addition of 5M sodium hydroxide aqueous solution to make the solution pH to be 8.9. This solution was maintained at a temperature equal to 34° C. and was then allowed to cool to 20° C.

Next, 250 mL of ethyl acetate was added and the liquid-liquid extraction step was conducted at 20° C. Impurities (including vanillyl alcohol and guaiacol) and small portion of vanillin were extracted into the organic phase. The aqueous phase containing mainly vanillin, ferulic acid and residual ethyl acetate. Ethyl acetate was then stripped under vacuum (0.5 bar) at 35° C. with injection of nitrogen for 1 hours.

Sulfuric acid (4M in water) was then added to the aqueous phase so as to obtain a solution with a pH of 6.4 at 30° C. A solid was then precipitated when temperature dropped to 20° C. and filtered off. The solid was washed twice with water. The solid was found to be vanillin with the purity of 95%. The overall yield of vanillin is >85%.

### Example 8 (Distillation Method)

In a prophetic example, 1000 g of crude vanillin solution produced according to Example 3 was neutralized to pH 7 and extracted with 1000 mL of ethyl acetate twice. The ethyl acetate layer was separated by a separatory funnel.

The ethyl acetate fraction was fully evaporated using a rotary evaporator at 65° C. under vacuum (100-500 mbar). The dried product was an orange/brown liquid oil.

The liquid oil was transferred to an appropriately sized round bottom flask to fit an available heating mantle with PID internal temperature control and magnetic stirring. A magnetic stir bar is added to the flask.

The round bottom flask is placed in the heating mantle set to 60° C. with magnetic stirring, and the condenser coolant is 20% propylene glycol in water at 5° C.

The round bottom flask is outfitted with heat tape to promote the evaporation of condensed vanillin on the walls of the flask. The temperature of the heat tape is set to the same temperature as the heating mantle throughout operation.

A short-path distillation condenser is attached to the round bottom flask, followed by a Dewar-type cold trap filled with salted ice to minimize vanillin losses.

A first fraction comprising of residual ethyl acetate and water was first recovered. The step of removing the ethyl acetate was ended when the operating pressure was 4 mbar for a heating mantle temperature of 80° C.

The temperature of the heating mantle is gradually increased to 110° C.

The distillation was stopped when the temperature at the top reached 125° C. The distillation yield was >90% vanillin after washing the condenser, cold trap, and connecting glassware with ethanol.

### Example 9 (Stripping Method)

In a prophetic example, 1000 g of crude vanillin solution (vanillin weight percentage around 15%) produced according to Example 3 was washed with sodium hydroxide aqueous solution.

A step (a) of distillation in the presence of water was carried out at a temperature of 80° C. under a pressure of 100 mbar. The stream at the outlet comprised of vanillin in around 80% weight percentage.

A step (b) of stripping was obtained on conclusion of step (a), at a temperature of 90° C. using, as entraining liquid, water which is vaporized under the operating conditions: liquid water was added at atmospheric pressure and then the pressure was lowered down to 25 mbar. The stream at the outlet comprised of vanillin in around 80% weight percentage.

The outlet stream was evaporated to achieve vanillin purity of 92% by weight.

Finally, the vanillin sample was purified via crystallization to achieve a titer of 99% and is obtained with a yield of 85%.

Having described various processes, systems, and compositions, certain aspects can include, but are not limited to:

In a first aspect, a biologically pure culture of Amycolatopsis tucumanensis LGM 24814 or a mutant thereof is claimed, which can convert ferulic acid to vanillin.

In a second aspect, a process for preparing vanillin comprises: subjecting ferulic acid to Amycolatopsis tucumanensis LGM 24814, or mutant thereof or an isolated enzyme thereof; converting at least a portion of the ferulic acid to vanillin, for a period of time sufficient to convert the ferulic acid to vanillin; and recovering the vanillin thus formed.

A third aspect can include the process of the second aspect, wherein the ferulic acid is natural ferulic acid.

A fourth aspect can include the process of the second or third aspect, wherein the ferulic acid is natural ferulic acid with a purity of less than 20%, or less than 30%, or less than 50%, or less than 90%, each by weight.

A fifth aspect can include the process of the second or third aspect, wherein the ferulic acid is natural ferulic acid of ≤20% purity or up to 60% purity is converted to vanillin forming a product mixture product containing 5-60% vanillin; 0-45% bioproducts comprising at least one of guaiacol, vanillic acid, vanillyl alcohol, 4-vinylguaiacol, protocatechuic acid, catechol, and ferulic acid; 0-70% lignin or lignin derived compounds; 0-60% carbohydrates, hemicellulose or carbohydrate derived materials; 0-30% protein, amino acids or protein derivatives; and 0-30% oils, fatty acids, fatty acid esters, sterols or phytosterols.

In a sixth aspect, a process comprises using vanillin as a flavoring, wherein the improvement comprises using as the vanillin the vanillin produced by a process according to any one of the second to fifth aspects.

A seventh aspect can include the process of the sixth aspect, wherein the vanillin is produced from natural ferulic acid.

An eighth aspect can include the process of any one of the second to seventh aspects, wherein the microorganism is used to convert ferulic acid to vanillin, and the microorganism is contained in a medium which comprises a carbon source.

A ninth aspect can include the process of the eighth aspect, wherein the carbon source is selected from the group consisting of sugars, sugar alcohols, organic acids and complex mixtures.

A tenth aspect can include the process of the eighth or ninth aspect, wherein the medium further comprises a nitrogen Source.

In an eleventh aspect, a process for preparing vanillin comprises: subjecting ferulic acid to Amycolatopsis tucumanensis LGM 24814, or mutant thereof or isolated enzyme thereof; converting the ferulic acid to vanillin for a period of time sufficient to convert the ferulic acid to vanillin, and recovering the vanillin thus formed, wherein the Amycolatopsis tucumanensis LGM 24814 or mutant thereof is contained in a medium which comprises a carbon source.

A twelfth aspect can include the process of the eleventh aspect, wherein the carbon source is selected from the group consisting of sugars, sugar alcohols, organic acids and complex mixtures, and optionally wherein the medium further comprises a nitrogen source.

In a thirteenth aspect, a process comprises using vanillin as a flavoring, wherein the improvement comprises using as the vanillin the vanillin produced by a process according to any one of the eleventh to thirteenth aspects.

In a fourteenth aspect, a process for preparing vanillin comprises: subjecting ferulic acid to Amycolatopsis tucumanensis LGM 24814, or mutant thereof or isolated enzyme thereof which converts ferulic acid to vanillin, for a period of time sufficient to convert the ferulic acid to vanillin, and recovering the vanillin thus formed, wherein the Amycolatopsis tucumanensis LGM 24814 or mutant thereof is contained in a medium which comprises a nitrogen source.

A fifteenth aspect can include the process of the fourteenth aspect, wherein the nitrogen source is selected from the group consisting of yeast extract, urea, ammonia, amino acids and complex mixtures.

In a sixteenth aspect, a process for preparing vanillin comprises: subjecting ferulic acid to Amycolatopsis tucumanensis LGM 24814, or mutant thereof or isolated enzyme thereof which converts ferulic acid to vanillin, for a period of time sufficient to convert the ferulic acid to vanillin, and recovering the vanillin thus formed, wherein the Amycolatopsis tucumanensis LGM 24814 or mutant thereof is contained in a medium which comprises antifoam agent (deformer).

A seventeenth aspect can include the process of the sixteenth aspect, wherein the antifoam agent is selected from the group consisting of organic non-silicon-based polymer, liquid silicon-based polymer, solid non-ionic polymer, fatty acids and neutral lipids natural oil, and mixture of natural fatty acids and triacylglycerols.

An eighteenth aspect can include the process of the sixteenth or seventeenth aspect, wherein the antifoam agent is added to the medium converts ferulic acid to vanillin with Amycolatopisis tucumanensis LGM 24814, or mutant thereof, or isolated enzyme thereof at concentration between 0.1 ppm to 300 ppm.

In a nineteenth aspect, a process for preparing vanillin comprises: subjecting ferulic acid to Amycolatopsis tucumanensis LGM 24814, or mutant thereof or isolated enzyme thereof which converts ferulic acid to vanillin, for a period of time sufficient to convert the ferulic acid to vanillin, and recovering the vanillin thus formed, wherein the Amycolatopsis tucumanensis LGM 24814 or mutant thereof is contained in a medium which comprises dissolved oxygen content.

A twentieth aspect can include the process of the nineteenth aspect, wherein the dissolved oxygen content in medium is maintained by 0-20 Hz (0-1200 rpm) agitation.

A twenty first aspect can include the process of the nineteenth or twentieth aspect, wherein the dissolved oxygen content in medium is maintained between 0-25 mg oxygen per liter of medium.

In a twenty second aspect, a process for preparing Vanillin comprises subjecting ferulic acid to the microorganism Amycolatopsis tucumanensis LGM 24814, or a mutant thereof or an isolated enzyme thereof which converts ferulic acid to vanillin, to convert the ferulic acid to the vanillin in a concentration of the vanillin of at least 1 g/l within a period of time from 4 to 120 hours, and recovering the vanillin thus formed.

A twenty third aspect can include the process of any one of the second to twenty second aspects in which vanillin and derivatives thereof are purified, starting with a fermentation broth, containing vanillin in water, containing impurities, comprising the following steps: a) a step of centrifugation of fermentation broth to remove cellular biomass/debris and particulate matter; b) a step of liquid/liquid extraction by placing the aqueous solution obtained after step a) in contact with a solvent E7, to obtain an organic phase containing vanillin and impurities, and an aqueous phase containing water-soluble impurities, salts, and residual solvent; c) a step concentrating the vanillin rich organic phase to yield a concentrated crude vanillin product; d) a step of distillation of the crude concentrated vanillin obtained from the evaporation of the organic phase in step c), and e) a step of multi-step recrystallization of the vanillin to yield a high purity vanillin.

In a twenty fourth aspect, a process for purifying vanillin and derivatives thereof, starting with an initial solution of vanillin or of a vanillin derivative in a solvent S1 containing impurities, comprises: evaporating the solvent S1 in the presence of water from the initial solution of the vanillin or of the vanillin derivative containing the solvent S1 to obtain an aqueous solution of the vanillin or of the vanillin derivative; using liquid/liquid extraction by placing the aqueous solution obtained after step a) in contact with a solvent S2, at a pH greater than 8 and less than 10, to obtain an organic phase comprising the solvent S2 and an aqueous phase containing the vanillin or the vanillin derivative and residual solvent S2;

precipitating, at a pH of between 4 and 7.5, the Vanillin or the derivative contained in the aqueous phase obtained after the liquid-liquid extraction, and isolating the vanillin or the derivative thereof.

A twenty fifth aspect can include the process of the twenty fourth aspect, wherein the solvent S2 is different from the solvent S1.

A twenty sixth aspect can include the process of the twenty fourth or twenty fifth aspect, wherein the solvent S1 has a boiling point of less than 100° C. or forms an azeotrope, with water, having a boiling point of less than 100° C.

A twenty seventh aspect can include the process of any one of the twenty fourth to twenty sixth aspects, wherein the solvent S1 is an organic solvent selected from the group consisting of alkyl acetates, (methyl ethyl ketone (MEK), cyclohexane, dichloromethane, and combinations thereof, or is a mixture of water and the organic solvent.

A twenty eighth aspect can include the process of any one of the twenty fourth to twenty seventh aspects, wherein the solvent S2 has a maximum solubility in water of 70 g/l.

A twenty ninth aspect can include the process of any one of the twenty fourth to twenty eighth aspects, wherein the solvent S2 is selected from the group consisting of dichloromethane; cyclohexane; ethyl acetate; propyl acetate; isopropyl acetatel; n-butyl acetate; isoamyl acetate; methyl isobutyl ketone (MIBK); butanol; mixture of dimethyl adipate, dimethyl succinate and dimethyl glutarate; and a mixture of these solvents.

A thirtieth aspect can include the process of any one of the twenty fourth to twenty ninth aspects, wherein the solvent S2 is isopropyl acetate.

A thirty first aspect can include the process of any one of the twenty fourth to thirtieth aspects, wherein the liquid-liquid extraction step comprises adding a weak base or a strong base.

A thirty second aspect can include the process of any one of the twenty fourth to thirty first aspects, wherein the liquid-liquid extraction step is performed at a pH of between 8.5 and 9.

A thirty third aspect can include the process of the thirtieth or thirty first aspect, wherein the weak or strong base is diluted in water to a concentration from 5% to 30% by weight relative to the weight of the aqueous solution in which the weak or strong base is diluted.

A thirty fourth aspect can include the process of any one of the twenty fourth to thirty third aspects, wherein the liquid-liquid extraction step is performed with a weight ratio between the solvent S2 and the vanillin from 0.2 to 3.

A thirty fifth aspect can include the process of any one of the twenty fourth to thirty fourth aspects, wherein the aqueous phase obtained after the liquid-liquid extraction step is subjected to a step for removing the residual solvent S2.

A thirty sixth aspect can include the process of any one of the twenty fourth to thirty fifth aspects, wherein the pH during the precipitating step is between 5.7 and 6.5.

A thirty seventh aspect can include the process of any one of the twenty fourth to thirty sixth aspects, wherein the isolating step consists of at least one step of recovering solid vanillin on a filter or spin-dryer, followed by one or more steps of washing with water.

A thirty eighth aspect can include the process of any one of the twenty fourth to thirty seventh aspects, further comprising, after the isolating step, recrystallizing the vanillin from water or from an alcohol/water mixture.

A thirty ninth aspect can include the process of any one of the twenty fourth to thirty eighth aspects, wherein the solvents S1 and S2 are identical.

A fortieth aspect can include the process of any one of the twenty fourth to thirty ninth aspects, wherein the initial solution comprises a weight content in vanillin from 10% to 60% by weight.

A forty first aspect can include the process of any one of the twenty fourth to fortieth aspects, wherein the initial solution comprises a weight ratio of impurities/vanillin between 0.10 and 0.35.

A forty second aspect can include the process of any one of the twenty fourth to forty first aspects, wherein the vanillin or the vanillin derivative in the aqueous solution obtained after the liquid-liquid extraction step is in a vanillate form, and wherein the aqueous solution obtained after the liquid-liquid extraction step contains impurities species whose pKa is below the pKa of vanillin.

A forty third aspect can include the process of any one of the twenty fourth to forty second aspects, wherein the impurities in the initial solution comprise benzoic acid, vanillyl alcohol, guaiacol, or mixtures thereof, and optionally further comprise species selected from the group consisting of vanillic acid, ferulic acid, dimers comprising two phenyl groups, dimers comprising a ferulic group, and heavy compounds; and wherein the liquid-liquid extraction step is carried out to separate some of these impurities by way of difference in their pKa from the vanillin.

A forty fourth aspect can include the process of any one of the twenty fourth to forty third aspects, wherein the impurities in the initial solution comprise lignin, carbohydrates, proteins, oils or derivatives of such; and wherein the liquid-liquid extraction step b) is carried out to separate some of these materials from vanillin.

In a forty fifth aspect, a natural vanillin has a purity greater than or equal to 96% that is in the form of an amorphous solid of which the color, in ethanolic solution at 10% by weight, is less than or equal to 200 Hazen.

A forty sixth aspect can include the natural vanillin of the forty fifth aspect which can be obtained by a process comprising at least one step in which natural vanillin is evaporated.

A forty seventh aspect can include the natural vanillin of the twenty third, twenty fourth, forty fifth, or forty sixth aspects having a purity greater than or equal to 98%.

A forty eighth aspect can include the natural vanillin of the forty sixth aspect, having a purity greater than or equal to 99% and containing less than 1000 ppm of vanillyl alcohol.

A forty ninth aspect can include the natural vanillin of any one of the forty fifth to forty eighth aspects, having a purity of 96% to 98.9% and comprising up to 3% by weight of impurities.

A fiftieth aspect can include the natural vanillin of any one of the forty fifth to forty ninth aspects, which is formed by direct solidification.

A fifty first aspect can include the natural vanillin of any one of the forty fifth to fiftieth aspects, which is refined by milling or sieving.

A fifty second aspect can include the natural vanillin of any one of the forty fifth to fifty first aspects, having a purity greater than or equal to 98%.

A fifty third aspect can include the natural vanillin of the fifty second aspect, having a purity greater than or equal to 99% and containing less than 1000 ppm of vanillyl alcohol.

A fifty fourth aspect can include the natural vanillin of any one of the forty fifth to fifty third aspects, having a purity of 96% to 98.9% and comprising up to 3% by weight of impurities.

In a fifty fifth aspect, a natural vanillin has a purity greater than or equal to 96% that is in the form of an amorphous solid of which the color, in ethanolic solution at 10% by weight, is less than or equal to 100 Hazen.

A fifty sixth aspect can include the natural vanillin of the fifty fifth aspect having a purity greater than or equal to 99%.

A fifty seventh aspect can include the natural vanillin of the fifty fifth or fifty sixth aspect which is formed by spalling, pelletizing or prilling, more preferably by spalling on a cylinder or on a belt.

A fifty eighth aspect can include the natural vanillin of any one of the fifty fifth to fifty seventh aspects, which is formed by spalling on a cylinder or on a belt.

A fifty ninth aspect can include the natural vanillin of any one of the fifty fifth to fifty eighth aspects, having a purity greater than or equal to 99%.

A sixtieth aspect can include the natural vanillin of the fifty ninth aspect, having a purity greater than or equal to 99% and containing less than 1000 ppm of vanillyl alcohol.

A sixty first aspect can include the natural vanillin of the fifty fifth aspect, having a purity of 96% to 98.9% and comprising up to 3% by weight of impurities.

In a sixty second aspect, a natural vanillin has a purity greater than or equal to 95%.

A sixty third aspect can include the natural vanillin of the sixty second aspect, which can be obtained by means of a process comprising one step in which organic solvent is used to precipitate salts impurities.

A sixty fourth aspect can include the natural vanillin of the sixty second or sixty third aspect, which can be obtained by means of a process comprising one step in which hot water (45 to 95° C.) is used to precipitate water insoluble impurities.

A sixty fifth aspect can include the natural vanillin of any one of the sixty second to sixty fourth aspects, which can be obtained by means of a process comprising one step in which organic solvent is used to extract vanillin out of aqueous solution.

In a sixty sixth aspect, a process comprises: separating a natural vanillin from a solution, wherein the natural vanillin has a purity greater than or equal to 96% that is in the form of an amorphous solid of which the color, in ethanolic solution at 10% by weight, is less than or equal to 200 Hazen.

A sixty seventh aspect can include the process of the sixty sixth aspect, wherein separating the natural vanillin comprises evaporating the natural vanillin.

A sixty eighth aspect can include the process of the sixty sixth or sixty seventh aspect, wherein the natural vanillin has a purity greater than or equal to 98%.

A sixty ninth aspect can include the process of any one of the sixty sixth to sixty eighth aspects, wherein separating the natural vanillin comprises direct solidification of the natural vanillin.

A seventieth aspect can include the process of any one of the sixty sixth to sixty ninth aspects, further comprising refining the natural vanillin by milling or sieving.

A seventy first aspect can include the process of any one of the sixty sixth to seventieth aspects, wherein separating the natural vanillin comprises using an organic solvent is used to precipitate salts impurities.

A seventy second aspect can include the process of any one of the sixty sixth to seventy first aspects, wherein separating the natural vanillin comprises a step in which hot water (45 to 95° C.) is used to precipitate water insoluble impurities.

A seventy third aspect can include the process of any one of the sixty sixth to seventy second aspects, wherein separating the natural vanillin comprises a step in which organic solvent is used to extract vanillin out of aqueous solution.

In a seventy fourth aspect, a process for the purification of natural vanillin resulting from a biotechnological process comprises: stripping a liquid stream comprising natural vanillin with an entraining gas and/or a vaporized liquid, in which the concentration by weight of natural vanillin in the liquid stream is greater than or equal to 10%, wherein a 10% by weight ethanolic solution of the natural vanillin obtained in the step of stripping the liquid stream comprising natural vanillin.

A seventy fifth aspect can include the process of the seventy fourth aspect, in which the concentration by weight of natural vanillin in the liquid stream is greater than or equal to 30%.

A seventy sixth aspect can include the process of the seventy fourth aspect, additionally comprising preparing the liquid stream of natural vanillin by evaporating, optionally in the presence of water, a solvent of a stream originating from the production of natural vanillin.

A seventy seventh aspect can include the process of the seventy fourth aspect, further comprising, after the step of stripping the liquid stream comprising natural vanillin with an entraining gas, removing the compounds which are less volatile than vanillin.

A seventy eighth aspect can include the process of the seventy fourth aspect, in which the entraining gas or the vaporized liquid is selected from the group consisting of water, steam, alkyl acetates, alcohols, inert gases selected from N2, CO2, He, Ar, and depleted air, and their mixtures.

A seventy ninth aspect can include the process of the seventy fourth aspect, in which the step of stripping the liquid stream comprising natural vanillin with an entraining gas is carried out at a temperature of greater than or equal to 20° C., and less than or equal to 140° C.

An eightieth aspect can include the process of the seventy fourth aspect, in which the step of stripping the liquid stream comprising natural vanillin with an entraining gas is carried out under an inert atmosphere.

In an eighty first aspect, a natural vanillin product resulting from a biotechnological process capable of being obtained according to the defined process of the seventh forth aspect, wherein the natural vanillin exists in the form of a solid.

An eighty second aspect can include the natural vanillin of the eighty first aspect, characterized in that it exhibits a conforming organoleptic profile.

An eighty third aspect can include the natural vanillin of the eighty first aspect, in which the concentration by weight of natural vanillin in the liquid stream is greater than or equal to 50%.

An eighty fourth aspect can include the natural vanillin of the eighty third aspect, wherein compounds less volatile than vanillin are removed in a vacuum film evaporator or in a thin film evaporator.

An eighty fifth aspect can include the natural vanillin of the eighty fourth aspect, in which the entraining gas or the vaporized liquid is water or steam.

An eighty sixth aspect can include the natural vanillin of the seventy fourth aspect, in which the step of stripping the liquid stream comprising natural vanillin with an entraining gas is carried out at a temperature greater than or equal to 30° C. and less than or equal to 120° C.

An eighty seventh aspect can include the natural vanillin of the eighty sixth aspect, wherein the step of forming natural vanillin comprises forming natural vanillin by crystallization.

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

1. A composition comprising:
a biologically pure culture of Amycolatopsis tucumanensis LGM 24814, a mutant thereof, or an enzyme extracted thereof;
ferulic acid; and
a carbon source.

2. A process for preparing vanillin comprising:
subjecting ferulic acid to Amycolatopsis tucumanensis LGM 24814, a mutant thereof, or an isolated enzyme thereof for a period of time sufficient to convert the ferulic acid to vanillin;
converting at least a portion of the ferulic acid to vanillin; and
recovering the vanillin.

3. The process of claim 2, wherein the ferulic acid is natural ferulic acid.

4. The process of claim 1, wherein the ferulic acid is natural ferulic acid with a purity of less than, or equal to, 90% by weight.

5. The process of claim 2, wherein the ferulic acid is natural ferulic acid of less than 60% purity, wherein the ferulic acid is converted to vanillin forming a product mixture product containing 0.5-60% vanillin; 0-45% bioproducts comprising at least one of guaiacol, vanillic acid, vanillyl alcohol, 4-vinylguaiacol, protocatechuic acid, catechol, and ferulic acid; 0-70% lignin or lignin derived compounds; 0-60% carbohydrates, hemicellulose or carbohydrate derived materials; 0-30% protein, amino acids or protein derivatives; and 0-30% oils, fatty acids, fatty acid esters, sterols or phytosterols.

6. The process of claim 2, further comprising:
using the vanillin as a flavoring.

7. (canceled)

8. The process of claim 2, wherein the Amycolatopsis tucumanensis LGM 24814, the mutant thereof, or the isolated enzyme thereof is contained in a medium which comprises a carbon source.

9. The process of claim 8, wherein the carbon source is selected from the group consisting of sugars, sugar alcohols, organic acids and complex mixtures.

10. The process of claim 8, wherein the medium further comprises a nitrogen source.

11.-15. (canceled)

16. The process of claim 10, wherein the nitrogen source is a group of inorganic nitrogen sources or organic nitrogen sources comprising yeast extract, urea, ammonia, ammonium salts, amino acids and complex mixtures.

17. The process of claim 2, wherein the Amycolatopsis tucumanensis LGM 24814 or mutant thereof is contained in a medium which comprises antifoam agent (deformer).

18. The process of claim 17, wherein the antifoam agent is selected from the group consisting of organic non-silicon-based polymer, liquid silicon-based polymer, solid non-ionic polymer, fatty acids and neutral lipids natural oil, and mixture of natural fatty acids and triacylglycerols.

19. The process of claim 17, wherein the antifoam agent is added to the medium converts ferulic acid to vanillin with Amycolatopisis tucumanensis LGM 24814, the mutant thereof, or the isolated enzyme thereof at concentration between 0.1 ppm to 300 ppm.

20. The process of claim 2, wherein the Amycolatopsis tucumanensis LGM 24814 or mutant thereof is contained in a medium which comprises dissolved oxygen content.

21. The process of claim 20, wherein the dissolved oxygen content in medium is maintained between 0-25 mg oxygen per liter of medium.

22. The process of claim 2, wherein converting at least the portion of the ferulic acid to vanillin comprises:
converting at least the portion of the ferulic acid to the vanillin in a concentration of the vanillin of at least 1 g/L within a period of time from 4 to 120 hours.

23. The process The process of claim 2, wherein a fermentation broth comprises the vanillin in water, and impurities, further comprising the following steps:
a) a step of centrifugation of fermentation broth to remove cellular biomass/debris and particulate matter;
b) a step of liquid/liquid extraction by placing the aqueous solution obtained after step a) in contact with a solvent E7, to obtain an organic phase containing vanillin and impurities, and an aqueous phase containing water-soluble impurities, salts, and residual solvent;
c) a step concentrating the vanillin rich organic phase to yield a concentrated crude vanillin product
d) a step of distillation of the crude concentrated vanillin obtained from the evaporation of the organic phase in step c), and
e) a step of multi-step recrystallization of the vanillin to yield a high purity vanillin.

24. A process for purifying vanillin and derivatives thereof, starting with the process of claim 2, wherein the vanillin is recovered in an initial solution of vanillin or of a vanillin derivative in a solvent S1 containing impurities, comprising the following steps:
evaporating the solvent S1 in the presence of water from the initial solution of the vanillin or of the vanillin derivative containing the solvent S1 to obtain an aqueous solution of the vanillin or of the vanillin derivative;
using liquid/liquid extraction by placing the aqueous solution obtained after step a) in contact with a solvent S2, at a pH greater than 8 and less than 10, to obtain an organic phase comprising the solvent S2 and an aqueous phase containing the vanillin or the vanillin derivative and residual solvent S2;
precipitating, at a pH of between 4 and 7.5, the Vanillin or the derivative contained in the aqueous phase obtained after the liquid-liquid extraction, and
isolating the vanillin or the derivative thereof.

25. The process of claim 23, wherein the impurities in the initial solution comprise lignin, carbohydrates, proteins, oils or derivatives of such; and wherein the liquid-liquid extraction step is carried out to separate some of these materials from vanillin.

26. The process of claim 23, wherein the impurities in the initial solution comprise benzoic acid, vanillyl alcohol, guaiacol, or mixtures thereof, and optionally further comprise species selected from the group consisting of vanillic acid, ferulic acid, dimers comprising two phenyl groups, dimers comprising a ferulic group, and heavy compounds; and wherein the liquid-liquid extraction step is carried out to separate some of these impurities by way of difference in their pKa from the vanillin.

27. (canceled)
